(19) United States
(12) Patent Application Publication
(10) Pub. No.: US 2022/0401672 A1
(43) Pub. Date: Dec. 22, 2022

(54) SYSTEMS AND METHODS FOR HYPOXIA

(71) Applicant: SPAULDING REHABILITATION HOSPITAL CORPORATION, Charlestown, MA (US)

(72) Inventors: Randy D. TRUMBOWER, Charlestown, MA (US); Jeremy J. PAPADOPOULOS, Charlestown, MA (US); Andrew Q. TAN, Charlestown, MA (US); Anthony N. CORSTEN, Charlestown, MA (US)

(21) Appl. No.: 17/771,328

(22) PCT Filed: Oct. 26, 2020
(86) PCT No.: PCT/US2020/057357

§ 371 Date: Apr. 22, 2022

Related U.S. Application Data
(60) Provisional application No. 62/925,306, filed on Oct. 24, 2019.

(51) Int. Cl. A61M 16/00; A61M 16/10; A61M 16/12; A61M 16/06; A61M 16/08; A61M 16/20; A61M 16/22

(52) U.S. Cl. CPC A61M 16/024; A61M 16/0045; A61M 16/1005; A61M 16/125; A61M 16/0066; A61M 16/0078; A61M 16/06; A61M 16/0833; A61M 16/107; A61M 16/204; A61M 16/22; A61M 2205/3368; A61M 2230/06

(57) ABSTRACT

Systems and methods for hypoxia delivery are provided. An apparatus for providing intermittent normoxia and hypoxia intervals includes a breathing component, a normoxia fluid source, a hypoxia fluid source, a valve, and a control system. The valve is configured to disrupt flow from at least one of the normoxia fluid source and the hypoxia fluid source and the control system is configured to cause the at least one valve to switch between delivery of fluid from the normoxia fluid source and the hypoxia fluid source while maintaining positive pressure at the breathing component.

### CROSS-REFERENCES TO RELATED APPLICATIONS

The present application is based on and claims priority to U.S. Provisional Patent Application No. 62/925,306, filed on Oct. 24, 2019, which is incorporated herein by reference in its entirety.

### STATEMENT REGARDING FEDERALLY SPONSORED RESEARCH

This invention was made with government support under HD 081274 awarded by the National Institutes of Health and under W81XWH-15-2-0045 awarded by the Department of Defense. The government has certain rights in the invention.

### BACKGROUND

Breathing a reduced concentration of oxygen can be a powerful biological factor in treating various sicknesses and injuries, and also in sports training. For some purposes it can be administered continuously for hours or even days. For example, professional endurance athletes will train at higher altitudes or expose themselves to low O₂ conditions using a hypoxicator generator to force their body to adapt to lower oxygen concentrations. As yet another example, a method referred to as acute intermittent hypoxia (AIH) has been utilized as part of a broader therapy for spinal cord injuries and other neurological problems.

In rodent models of spinal cord injuries, AIH triggers mechanisms of neural plasticity that enhance respiratory and non-respiratory function. AIH elicits long-term facilitation of phrenic motor neuron activity via a signaling cascade that up-regulates brain-derived neurotropic factor (BDNF) and subsequently initiates downstream cellular events that purportedly strengthen synapses between pre-motor and motor neurons. In experiments, rodents have been exposed to daily AIH for 7 consecutive days via a Plexiglas chamber flushed with 10, 5 min episodes of 10.5% O₂; 5 min intervals of 21% O₂, as described by Lovett-Barr, M. R., Satriotomo, I., Muir, G. D., Wilkerson, J. E., Hoffman, M. S., Vinit, S., Mitchell, G. S., 2012. Repetitive intermittent hypoxia induces respiratory and somatic motor recovery after chronic cervical spinal injury. J Neurosci 32, 3591-3600.

Clinical trials have modified existing air delivery technologies, such as traditional gas-delivery masks coupled to a dedicated canister of low-O₂ gas to administer AIH interventions, where the person could take the mask off to receive normal O₂ levels. Others have used commercially available pressure swing adsorption scrubber (PSA)-dependent generators, such as hypoxicator generators that are marketed to athletes for endurance training.

Thus, there is a continuing need for systems and methods that facilitate the delivery of controlled intermittent low O₂ conditions for clinical or medically-therapeutic settings, where it is paramount to meet the specific control, calibration, and functionality needed to assist the patient or subject with achieving an effective therapeutic outcome.

### BRIEF SUMMARY

The present disclosure provides systems and methods for a hypoxia therapy, including acute intermittent hypoxia therapy, or conditioning that overcomes the aforementioned drawbacks.

In one aspect, the present disclosure provides an apparatus for providing intermittent normoxia and hypoxia intervals to a subject. The apparatus includes a breathing component, a normoxia fluid source, a hypoxia fluid source, a manifold, at least one valve, and a control system. The breathing component can be configured to engage the subject to deliver at least one fluid to the subject for breathing. The normoxia fluid source can be coupled to the breathing component. The hypoxia fluid source can be coupled to the breathing component. The manifold can be fluidly coupled to the breathing component and arranged between the normoxia fluid source and the breathing component and the hypoxia fluid source and the breathing component to deliver fluid from the normoxia fluid source to the breathing component and deliver fluid from the hypoxia fluid source to the breathing component. The at least one valve can be configured to disrupt a flow of fluid from at least one of the normoxia fluid source and the hypoxia fluid source. The control system can be configured to cause the at least one valve to switch between delivery of fluid from the normoxia fluid source and the hypoxia fluid source while maintaining a positive fluid pressure at the breathing component over a full range of breathing by the subject.

In another aspect, the present disclosure provides a hypoxia delivery system. The system includes a breathing component, a subject monitoring system, a normoxia source, a hypoxia source, a first hose line, a second hose line, a valve system, and a controller. The breathing component can be configured to engage a face of a subject. The subject monitoring system can include a sensor configured to track a physiological parameter of the subject. The first hose line can be in fluid communication with the breathing component and the normoxia source. The second hose line can be in fluid communication with the breathing component and the hypoxia source. The valve system can be configured to control fluid flow from the normoxia source through the first hose line to the breathing component or from the hypoxia source through the second hose line to the breathing component. The controller can be in communication with the subject monitoring system and configured to control operation of the valve system using feedback from the subject monitoring system.

In another aspect, the present disclosure provides a method for providing intermittent normoxia and hypoxia intervals to a subject. The method includes securing a breathing component to the face of the subject, the breathing component including a hose manifold and at least one fluid sensor. The method also includes providing a normoxia fluid source, connecting the normoxia fluid source to the hose manifold, providing a hypoxia fluid source that is configured to provide a predetermined concentration of oxygen, and connecting the hypoxia fluid source to the hose manifold. The method also includes sensing at least one property of inspiratory and expiratory fluid from the subject with the at least one fluid sensor, and controlling normoxia and hypoxia control valves that are in fluid communication with the respective normoxia and hypoxia fluid sources to provide at least one period of normoxia fluid supply to the subject and at least one period of hypoxia fluid supply to the subject.

### BRIEF DESCRIPTION OF DRAWINGS

FIG. 1 is a schematic illustration of a hypoxia delivery system according to aspects of the present disclosure.

FIG. 2 is a schematic illustration of a blower according to aspects of the present disclosure.

FIG. 3 is a schematic illustration of a constant pressure reservoir according to aspects of the present disclosure.

FIG. 4 is another schematic illustration of a hypoxia delivery system according to aspects of the present disclosure.

FIG. 5 is a schematic illustration of a computer based control system according to aspects of the present disclosure.

FIG. 6A is a graph showing a change in O₂ during manual and automated switching between hypoxia and normoxia intervals.

FIG. 6B is a graph showing a change in O₂ during automated switching between hypoxia and normoxia intervals.

FIG. 6C is a graph showing cycle consistency between twelve hypoxia and normoxia intervals.

FIG. 7A is a graph showing oxygen concentration versus steady flow rate.

FIG. 7B is a graph showing a FiO₂ increase from breathing.

FIG. 7C is a graph showing FiO₂ during gentle-breathing hypoxia.

FIG. 8A is a graph showing fluctuations during hypoxia delivery.

FIG. 8B is a graph showing reduced fluctuations during hypoxia delivery.

FIG. 9 is a flowchart illustrating a method for using a hypoxia delivery system according to aspects of the present disclosure.

FIG. 10A is a schematic illustration of acute intermittent hypoxia (AIH) system according to aspects of the present disclosure.

FIG. 10B is a schematic illustration of another AIH system according to aspects of the present disclosure.

FIG. 11A is a graph showing time-dependent changes in relative O₂ in one experiment.

FIG. 11B is a graph showing time-dependent changes in relative O₂ in another experiment.

FIG. 12 illustrates a graphical representation of cumulative temporal errors in an AHI system.

FIG. 13 is a graphical representation of a relationship between delivered O₂ concentrations and pressure-swing absorption flow rate.

FIG. 14A is a graph of oxygen as a percent of ambient air over time in one experiment.

FIG. 14B is a graph showing the effects of a mixing chamber.

FIG. 15 is a graphical representation of temporal changes in blood oxygen saturation.

### DETAILED DESCRIPTION

Acute Intermittent Hypoxia (AIH) (for example, 90 seconds hypoxia intervals of 10% FiO₂ alternating with 60 seconds normoxia interval of approximately 20.8% FiO₂) has generally been shown to enhance motor recovery in persons with intramedullary spinal cord lymphoma (iSCL). Optimizing the IH dose to elicit beneficial neural plasticity without triggering pathology is a topic of considerable research. There is growing empirical evidence that brief, repeated exposures to hypoxia (in particular, acute intermittent hypoxia) safely induces functional recovery in persons with chronic, incomplete spinal cord injury in both non-respiratory and respiratory motor systems. Furthermore, when coupled with traditional rehabilitation training, low-dose AIH can further enhance recovery of motor functions in persons with iSCL. For example, AIH can be combined with weight-supported treadmill walking.

Understanding how hypoxia dose parameters interact with various physiological systems to achieve beneficial neuroplasticity without attendant pathology is an important topic of current research. Despite the lack of IH paradigm standardization across laboratories, AIH studies in humans have converged on a safe and effective dosing involving exposure to relatively ‘mild’ severities (such as approximately 9% to 10% FiO₂, for example) for approximately 90 seconds alternating with 60 seconds of normoxia for fifteen full cycles totally approximately 37.5 minutes. Such dosing has been documented to effectively promote functional recovery without such negative symptoms as cognitive impairments, episodes of autonomic dysreflexia or hypertension.

On the other hand, more severe (such as less than 9% FiO₂, for example) and chronic hypoxic exposures (such as over 100 cycles, for example) can potentially elicit negative effects. Optimizing IH as an adjuvant for neurorehabilitation therefore requires determining the greatest therapeutic effect that avoids triggering pathology. In general, research efforts addressing such balance require precise delivery methods for a wide range of controlled and repeatable AIH protocols delivered with a high safety of margin.

Some methods of AIH rely on manually timed connection and disconnection of a hypoxic-air supply, which may produce inconsistent dosing intervals and delivered fraction of inspired oxygen (FiO₂) values. In general, human error can lead to timing deviations, imprecision, and logging errors. Additionally, some scrubbers manufactured for athletic training may not deliver constant FiO₂: flow variations from subject respiration and/or plumbing restrictions, as well as internal variable scrubber processes can cause FiO₂ variation up to several percentage points of O₂ concentration.

In some methods, individual respiratory flow variations of an AIH method may overcome the positive pressure supplied by commercially available hypoxia sources, resulting in possible discomfort and raised FiO₂. Generally, translating AIH to the clinic requires the development of an automated delivery device that supports delivery consistency across multi-site clinical trials.

One method to improve motor function after SCI is through mild episodes of breathing low oxygen (i.e., acute intermittent hypoxia). As described briefly above, in rodent SCI models, AIH triggers mechanisms of neural plasticity that enhance respiratory and non-respiratory function. AIH elicits long-term facilitation of phrenic motor neuron activity via a signaling cascade that up-regulates brain-derived neurotropic factor (BDNF) and subsequently initiates downstream cellular events that purportedly strengthen synapses between pre-motor and motor neurons. One study exposed SCI rodents to daily (7 consecutive days) AIH via Plexiglas chamber flushed with 10, five minute episodes of 10.5% O₂; 5 min intervals of 21% O₂. They reported that these rodents substantially improved breathing capacity, as well as locomotor skills. More recently, results from clinical studies showed AIH-induced improvements in motor behaviors in persons with motor-incomplete SCI. In three separate studies, persons with iSCI showed increased ankle strength after a single day of AIH treatment that persisted for hours. Some studies have also uncovered substantial benefits of multi-session (up to 14 days) AIH on walking speed and endurance, as well as hand function.

Early clinical trials modified existing air delivery technologies to administer AIH interventions in persons with SCI. These AIH protocols consisted of 15, 60-90 second episodes of 10.0% O₂ along with 60 second intervals of ambient room air (20.9% O₂). These seminal studies involved or pressurized gas cylinders with programmable mechanical valve controls. Pressurized gas cylinder systems are well calibrated for laboratory application, but they are less feasible for clinic and home use due, in part, to stringent storage requirements and high maintenance costs, and the need for a specialist to administer and replenish gas from pressurized cylinders. The PSA-dependent delivery systems appear more practical since they have a smaller “footprint”, low maintenance costs, and minimal storage requirements; however, their inefficiencies in administering AIH requires further attention.

As briefly discussed above, breathing a reduced concentration of oxygen can be a powerful biological factor in treating various sicknesses and injuries, and also in sports training. For some purposes it can be administered continuously for hours or even days, in a procedure called Simulated Altitude Training. For other purposes (including sports training) an intermittent protocol (Intermittent Hypoxia Training, IH or IHT) is valuable: around 5 minutes of reduced oxygen (hypoxia, perhaps FiO₂˜10%) followed by approximately 5 minutes of normal oxygen (normoxia, FiO₂>20%), the sequence repeated multiple times. A protocol that can be valuable for recovery of spinal cord injury is Acute Intermittent Hypoxia (AIH). In this case the hypoxic and normoxic intervals are short (on the order of order 1 minute, for example), and an important aspect is the suddenness of concentration change (namely within a few seconds, preferably within 1 second). One example use of the technology is directed toward AIH, but may be also or instead useful for administering IH/IHT.

Some conventional methods of delivering Acute Intermittent Hypoxia can use pressurized gas cylinders and computer-controlled valves. High-pressure cylinders of precisely characterized gases are generally expensive for long term use, and inconvenient to replace. In addition the delivered flow rate is far too low to guarantee comfort when a subject feels a need to breathe deeply. Examples of the present disclosure include an AIH delivery system that avoids the gas-cylinder problems.

In some conventional methods, either a pressure swing adsorption scrubber, or membrane separation scrubber (sometimes called a hypoxicator) can be used to produce hypoxic gas conveniently and inexpensively. The produced gas can be manually connected to or disconnected from a user mask. The manual timing of this approach can be inconsistent, and breathing can be restricted in the disconnected state because there is no blower to help the subject inhale through the one-way mask inlet valve and associated tubing. Additionally, a normoxic valve positioned directly adjacent to the mask can be heavy and uncomfortable for the user. Furthermore, the scrubber output shows inconsistent oxygen concentration in its output, partly a ripple due to scrubber internal workings and partly a slow drift of oxygen concentration when the hose is disconnected, which will then take time to reverse during normoxia. Examples of the present disclosure can include features that facilitate accurate timing, comfortable breathing of normoxia, and more-consistent delivered FiO₂ during hypoxia.

Some conventional methods of IHT can use a scrubber, along with computer-controlled switching valves that cannot deliver a rapid change in air concentration to the subject because the intervening tubes and chambers must be cleared first. In other methods, a rebreathing chamber can be used to deplete oxygen, but this too leads to long concentration-switching times. Examples of the present disclosure can improve on the rapidity of concentration change provided by these and similar methods.

Some conventional hypoxia delivery systems include a single Hypoxico 123 scrubber (an athletic “high altitude” training product) that can be either set to minimum FiO₂ (assumed to be about 10%), or is adjusted to deliver an electrochemical sensor FiO₂ reading of 10%. A CPAP-style output hose can be manually connected to, or disconnected from (thus allowing room-air inspiration), a subject's dual-valve CPAP face mask. A timer readout can be employed to generate the desired dosing intervals of 90 s hypoxia, 60 s normoxia. The experimenter can manually log fingertip SpO₂, HR, and BP, as displayed by a GE Dash 4000 patient monitor, for example; and apply safety logic to terminate hypoxia administration or lengthen normoxia administration, if SpO₂ falls below certain limits. While the hose is connected to the scrubber, hypoxic air is delivered with positive mask pressure; but when it is disconnected, normoxic breathing requires slight inspiratory effort due to pressure drop from the mask inlet valve. In addition, the actual FiO₂ delivered to the subject, and the precise delivery times, are not accurately measured. In both manual and automated hypoxia delivery, each Hypoxico 123 scrubber can be equipped with the F-tube flow diverter for delivering lowest FiO₂. The scrubber FiO₂ setting is not computer controlled: rather, a valve is manually adjusted, and after a few minutes the delivered FiO₂ settles to a value correlated with the output flow. The scrubbers exhibit limitations as to delivery flow rate and concentration consistency at FiO₂=10%. The scrubber output passes through a HEPA filter, then is buffered by two 3L rubber reservoir bags to permit occasional or temporary hypoxia inspiration rates above scrubber output. Plumbing can be the standard CPAP wire-reinforced 19mm ID hose, with 22mm OD tapered rubber terminations, and various connectors, adapters, and tee fittings. Air can be delivered to a subject wearing a StarMed dual-valve CPAP mask, generally in conditions of continuous positive pressure except when a subject's deep breaths outpace the gas delivery.

As will be described, the present disclosure provides a generally cost-effective and easy-to-use apparatus for accurately and comfortably providing a human subject with scheduled bouts of hypoxia, interspersed with bouts of normoxia. The apparatus may be a relatively simple system that can be used in clinic or home settings. In some non-limiting examples, the scheduled bouts of hypoxia may be ten to twenty bouts with 90 seconds of air with oxygen reduced to the 10% level and the bouts of normoxia may be 60 seconds of atmospheric air with at least 20% oxygen. The changes in oxygen concentration presented to the subject occur rapidly. In some non-limiting examples, the changes in oxygen can occur in less than approximately 1 second to less than approximately 10 seconds.

In some examples, a hypoxic gas can be supplied by a pressure swing adsorption scrubber and possibly augmented by a mix chamber, with output concentration stabilized by shielding it from flow variations both when switching from normoxia and when a subject breathes deeply. The apparatus can supply constant positive pressure to a breathing component, such as a mask, for example by using a hypoxia reservoir during hypoxia intervals and by using a blower during normoxia intervals. The requisite sudden changes in concentration may be assured by using rapid-acting solenoid gas valves and dual hoses to the mask. In one example, a computer can be used to log valve actions, delivered gas concentration, and subject blood oxygen concentration. In general, the control system can automatically log all physiological data and sensor data to a drive which, in some instances, can eliminate the need for an administrator to constantly monitor a physical recording. A computer can also control the valves at scheduled times, and implement safety rules (e.g., altering normoxia duration as needed).

In some hypoxia delivery systems according to aspects of the present disclosure, the delivery system can support programmable interval timing to control instantaneous (or near instantaneous) gas switching via solenoid valves, smoother O₂ concentration delivery at positive pressure, and biosignal monitoring and logging for switching feedback. In some examples, the feedback is closed loop feedback. The system can include slow electrochemical and fast fiber optic FiO₂ sensors to measure delivered oxygen. In general, the system can provide relatively consistent interval lengths, reduce or eliminate observed systematic hypoxic duration errors that exceed 2 seconds, and deliver more consistent steady state FiO₂. Additionally, the system may be qualitatively more comfortable for the subject with increased positive pressure of one or more of the hypoxia or normoxia supply.

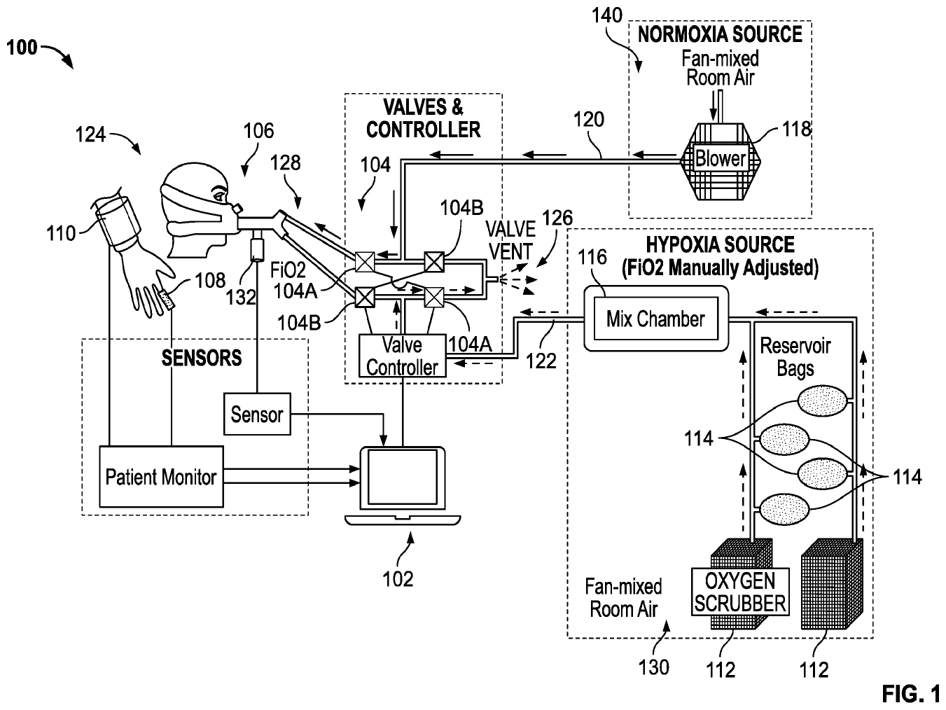

FIG. 1 illustrates a non-limiting example of a hypoxia delivery system 100 according to the present disclosure. The system 100 includes a computer 102 that can provide control to fast-acting valves 104 and a subject monitoring system. The subject monitoring system can monitor biosignals such as FiO₂ levels measured at a breathing component, oxygen saturation and heart rate measured via a fingertip sensor 108, and blood pressure measured via a blood pressure monitor 110. By way of example, in the illustrated embodiment, the breathing component is configured as a mask 106; however, other breathing components are possible, such as a cannula, for example. The system 100 further includes scrubbers 112, reservoir bags 114, a mix chamber 116, and a blower 118 configured to blow room air. By way of example, in the illustrated embodiment, the system includes dual scrubbers 112; however, other combinations of scrubbers are possible. Additionally, by way of example, the total volume of the reservoir bags 114 is approximately 12 L and the volume of the mix chamber is about 6L. In other embodiments, the system 100 can include one or more reservoir bags that may have a volume of less than 12 L or greater than 12 L. Likewise, in other embodiments, the system 100 can include a mix chamber that may have a volume greater than 6 L or less than 6 L.

Further illustrated in FIG. 1, the valves 104 includes a first pair of valves 104A and a second pair of valves 104B. The first valves 104A can direct airflow in the blower line 120 and the second valves 104B can direct airflow in the scrubber line 122. In particular, the first valve 104A can direct airflow from the blower 118 to a subject 124 or the first valve 104A can release air from the blower line 118 via a valve vent 126. Likewise, the second valve 104B can direct airflow from the scrubber line 122 to the subject 124 or the second valve 104B can release air from the scrubber line 122 via the valve vent 126.

By way of example, the solid arrows of FIG. 1 represent an operating state where the first valve 104A is open (e.g., energized) and the second valve 104B is closed (e.g., de-energized) thereby providing airflow from the blower 118 to the subject 124. When the airflow travels from the blower 118 to the subject 124, the subject 124 receives non-hypoxic air. Correspondingly, the dashed arrows of FIG. 1 represent an operating state where the second valve 104B is open and the first valve 104A is closed thereby providing airflow from the scrubbers 112 to the subject 124. When the airflow travels from the scrubbers 112 to the subject, the subject 124 receive hypoxic air.

The mask 106 includes a manifold configured as two hoses 128 that, for example, can be configured as CPAP hoses that are distinct. In other examples, the manifold can be configured as a series of valves. In use, the hoses 128 remain filled with gas of the appropriate concentration, and their flow is simply stopped or started via the valves 104. With the pre-filled (i.e., constant filled) hoses 128, there is virtually no delay between a valve command and delivery of the desired concentration at the mask 106. Additionally, the mix chamber 116 is used to smooth pulsatile variations in FiO₂ delivered from the scrubber 112. In general, the dual scrubbers 112 and the four reservoir bags 114 guarantee comfort and minimize disturbance to FiO₂ when the subject 124 takes occasional deep breaths. In some non-limiting examples, the mask 106 or other breathing components may be selectively detachable from the hypoxia delivery system 100, disposable, and replaceable.

The scrubbers 112, the reservoir bags 114, and the mix chamber 116 are part of a general hypoxia source 130. In some non-limiting examples, the hypoxia source 130 includes the two scrubbers 112 that are configured as Hypoxico 123 scrubbers with F-tube flow restrictors. Each scrubber 112 can be manually adjusted to deliver FiO₂ and approximately 10% (or sham=20.9%) after warmup. Additionally, in some non-limiting examples, the hypoxic source 130 includes the reservoir bags 114 that are configured as four 3-liter reservoir bags which allow the subject 124 to breathe deeply and comfortably. Further, in some non-limiting examples, the hypoxic source 130 includes the mixing chamber 116 that is configured as a 6-liter gas mixing chamber that is added between the scrubbers 112 and the valves 104. The mixing chamber 116 can minimize 0.5 Hz output ripple in the scrubber 112 FiO₂. The mixing chamber 116 can be formed from a generally cylindrical container having a screw port on each end. In some examples, the screw ports can include threaded caps with adapter nozzles that fit standard CPAP tubing. Fan-mixed room air is presented to the scrubber 112 inlets to reduce the chance that concentrated O₂ from the scrubber 112 may affect delivered FiO₂.

The blower 118 is part of a general normoxia source 140. The normoxia source 140 is configured to push room air through the plumbing (e.g., hoses 128) to the mask 106 with the normoxia source 140 delivery pressure regulated by a variable transformer. In some non-limiting examples, the voltage of the transformer can be set to approximately 20V AC to reduce cooling and drying sensations of high airflow. Continuous positive mask pressure is maintained during both hypoxia and normoxia for easy breathing including during deep breaths.

Still referring to FIG. 1, the valves 104 are configured as solenoid gas valves that connect the mask 106 to the respective hypoxia source 130 and normoxia source 140. In the illustrated example, the valves 104 are configured as four one-way valves; however, in other examples, other valve types and combinations may be possible, such as two two-way valves or a single four-way valve. Each of the valves 104 opens or closes its respective low-resistance orifice in milliseconds. In the illustrated example, at any instant, valves on one diagonal of the valves installment will both be open, while those on the other diagonal will both be closed. The valves are controlled to the second by a microcontroller, such as Arduino-actuated relays, commanded by a computer program from the graphical user interface (GUI) of the computer 102. In general, the computer 102 acts as a system controller that is in communication with the subject monitoring system and controls valve operation.

In one non-limiting example, signals from the fingertip sensor 108, the blood pressure monitor 110, and the heart rate monitor are captured by a conventional Masimo Root Platform (Maximo, Irvine Calif. USA) patient monitor. Just before the mask 106 is a FiO₂ sensor 132 which can be configured as a Maxtec OM25-RME electrochemical sensor (Maxtec, Salt Lake City, Utah 84119) with serial output. The device exhibits 12-second settling time and uses only single-point calibration. In some instances, it may be useful to calibrate the sensor to match atmospheric FiO₂ at 20.9% (or slightly less due to humidity). In some cases, a FiO₂ sensor may not be accurate near the 10% therapeutic value. In some examples, the FiO₂ sensor can be a Presens single channel fiber optic oxygen transmitter (Oxy1-ST, Regensberg, Germany) along with the corresponding glass fiber oxygen sensor (PM-PsT7) and temperature probe (Pt100).

The computer 102 can include an automated system that has three functions that can be controlled by a program, such as a custom C# program, for example. The first function is logging time-stamped valve-switching events and polling the MaxTec (for breathing component FiO₂) and Masimo (for SpO₂, HR, and BP) for 1 Hz logging. The second is sending commands to the Arduino-based valve relays. For example, valve 104A opens and valve 104B closes for 90 seconds (hypoxia). The third significant function of the computer is applying a safety-related valve-control rules: For example, if fingertip SpO₂ drops below 70% during hypoxia, a 60 second normoxia interval is immediately begun. If SpO₂ has not risen to 80% at the end of a normoxia interval, an additional 60 second normoxia interval is initiated. In other examples, the SpO₂ thresholds may be different and the time intervals may be different.

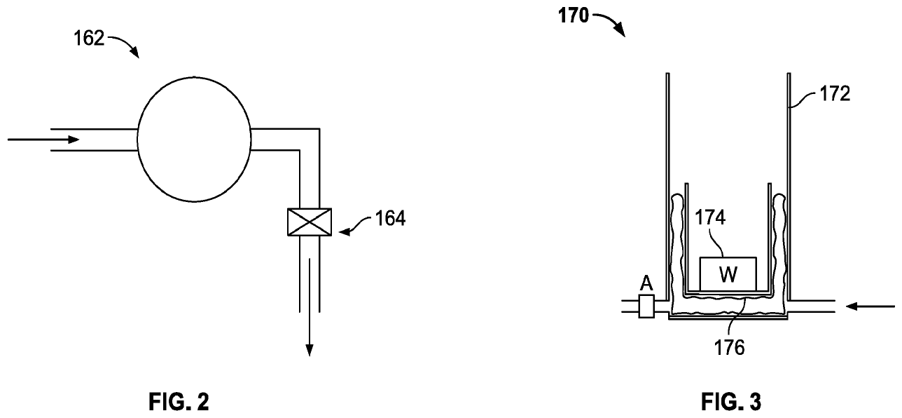

The blower 118 of FIG. 1 includes a vent valve to vent normoxic air back into the atmosphere; however, in other embodiments, a blower may only include a single valve. For example FIG. 2 illustrates a blower 162 having a single valve 164 which can eliminate a vent valve. The single valve 164 of the blower 162 generally reduces blower cooling but it can reduce cost of a blower overall. As a result, in some non-limiting examples, the hypoxia deliver system 100 can include the blower 162.

FIG. 3 illustrates an example of a constant pressure reservoir 170 according to some examples of the present disclosure. The constant pressure reservoir 170 includes a large container 172 open at the top, with a loose-fitting weighted plug 174. The space below and around the plug 174 is lined by a plastic bag 176 to trap air in a pressurized volume. The bag 176 acts as a rolling diaphragm as the plug 174 rises or falls—equivalent to a low-friction seal. The weight W is based on the plan area of the plug 174, in order to develop about 0.1 psi air pressure in the pressurized volume. For example if the plug area is about 0.25 m², a weight W=70 kg is needed. The pressurized volume can function with one fluid port serving both inlet and outlet needs. But two are drawn, to suggest that incoming air must pass through the reservoir, improving mixing to deliver uniform FiO₂. (In fact, in some examples, it may even replace a mix chamber). In use of this example version, a scrubber will continuously inject hypoxic air, causing the plug to rise. The weight W dictates the air pressure, which does not change: it is just correct to assist inhalation, and allows the scrubber to operate consistently with no pressure variation. When it is time to administer hypoxia, valve A opens to send gas through the hypoxia line to the breathing component (e.g., a mask).

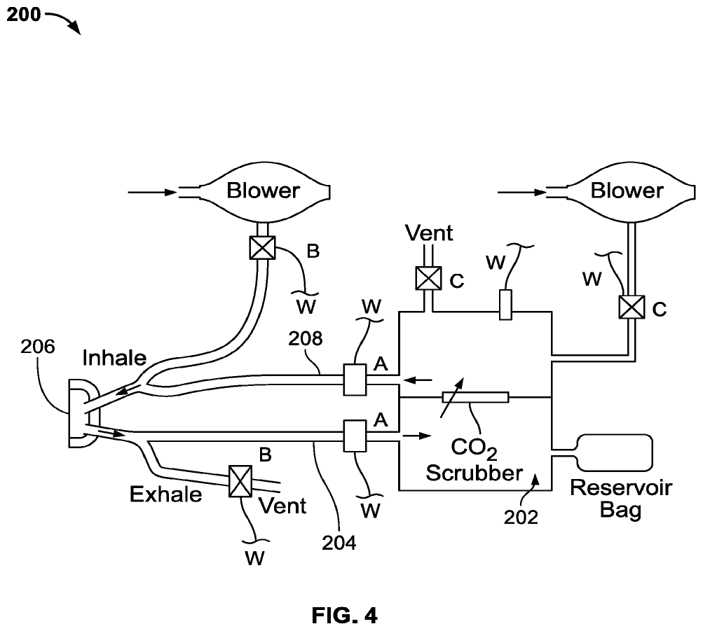

FIG. 4 illustrates a non-limiting example of an acute intermittent hypoxia system 200 that includes a rebreathing chamber 202. The chamber includes two parts with a dividing wall. Air can be exhaled through a manifold including a lower hose 204 (the control of which hose is used depends on the one-way valves in a mask 206) to the lower chamber of the rebreathing chamber 202. It passes through the CO₂ scrubber and possible desiccator to emerge in the upper chamber with reduced oxygen. That is what is breathed through an upper hose 208 of the manifold. After a certain period of rebreathing the same air, its FiO₂ is reduced. To prevent further reduction, an oxygen sensor with wire W that is read by a computer (not shown) causes the computer to briefly open valves C and admit a little room air while expelling some mixture. In summary, with A valves open the subject is immediately exposed to hypoxic air, and any inhaled air is returned to the rebreathing unit 202 for drying, CO₂ scrubbing, and a little oxygen addition if needed.

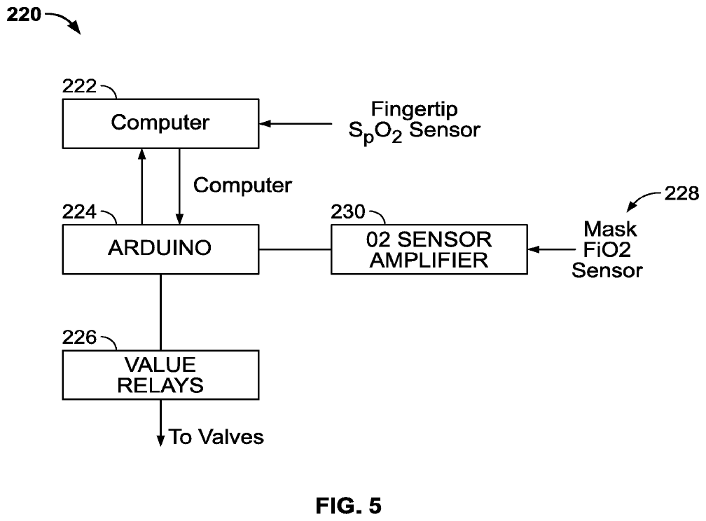

FIG. 5 illustrates a non-limiting example of a computer-based control system 220. The control system 220, in some examples, can be used with the hypoxia deliver system 100. The control system 220 includes a compute computer 222 that can communicate with a microcontroller 224, such as an Arduino Uno, for example, to give valve operation commands 226, and read data from the O₂ sensor 228. The computer also logs all information as to valve operation, data on FiO₂ 230 of gas entering the mask, and SpO₂ (blood oxygen from fingertip sensor). With the SpO₂ information it can alter hypoxia administration if needed for safety. The control system 220 can also include an O₂ sensor amplifier 232 in communication with the FiO₂ sensor 230.

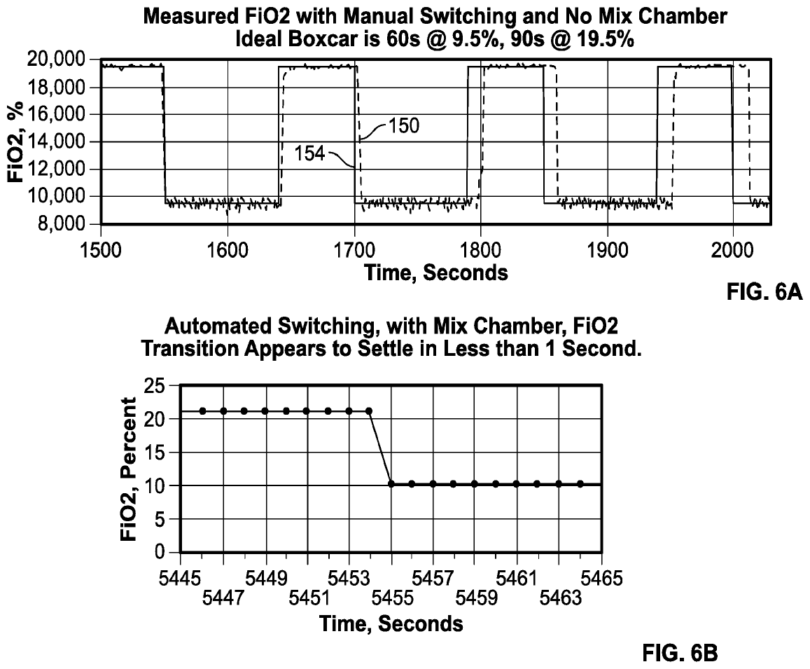

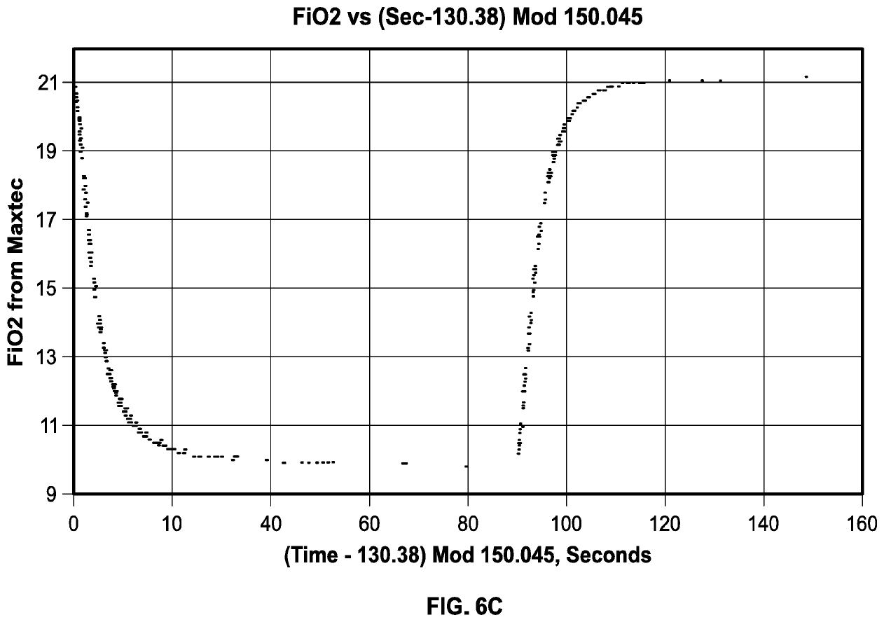

In general, the automated switching of the hypoxia delivery system 100 achieves both quick switching between gas intervals as well as consistency between the lengths of the intervals. FIG. 6A illustrates a plot of the delivery system 100 due to experimenter manual switching (line 150) versus the periodic ideal FiO₂ signal (line 154). The line 150 reveals errors in hypoxic/normoxic intervals that arise from manual switching. This demonstrates that manual timing is potentially imprecise and vulnerable to timing error by the experimenter. It can be noted that within the manual switching protocol, normoxic air is “supplied” to a participant by the experimenter simply disconnecting the CPAP hosing supplying hypoxic air from the mask. The only mask inflow is therefore generated by the subject inspiration alone. This breathing delay gives an appearance of delayed switching.

In some examples of hypoxia delivery systems, one or two oxygen scrubbers (hypoxicators) are each able to deliver at least 500 mL/s when output FiO₂ is set to 10%. As shown in FIG. 1, two Hypoxico scrubbers 112 with the F-tube diversion can facilitate breathing comfort for even a deep-breathing subject. Such scrubbers 112 can push air through masks so ordinary inspirations are made under continuous positive pressure. That is, more fluid flow is available to the subject at any given moment than the subject is consuming via inhalation.

In general, in a hypoxia delivery system, such as the system 100, reservoir bags (two each of the 3L size) can be T-connected to each scrubber output line to serve as reservoirs. These elastomeric balloons react with very slight pressure until 90% full, then rapidly climb in pressure to match scrubber output. When a deep-breathing subject inhales faster than the scrubber output, the difference comes from the reservoir bags. Since those bags are quickly lowered to near-zero pressure, at that point breathing becomes moderately harder for the subject. But only if the bags are fully depleted does the subject apply suction to the scrubber output—this dramatically increases scrubber output FiO₂. With four bags total it can be difficult for such suction to be applied by most subjects.

Another example reservoir (such as the reservoir 170 of FIG. 3) can include a chamber whose roof floats freely up and down, supported below by reservoir pressure, and pressed down by a fixed weight of about 70 kg/m². A water seal or rolling diaphragm seal can be engineered to exert very little vertical force, meaning reservoir pressure is defined by that weight. With such a reservoir, one scrubber may suffice, because instead of venting to atmosphere during normoxia supply to the subject, the scrubber would continue to fill the reservoir. Thus over an entire treatment made of 15 cycles of 90-second hypoxia and 60-second normoxia, the flow rate available to the subject during hypoxia intervals is 5/3 the flow rate of the scrubber alone. The scrubber output would not experience flow disturbance (hence FiO₂ disturbance) during normoxia. And the user will have greater deep-breathing comfort than provided by the 3L bags.

In some examples the output of a scrubber or scrubbers can be fed through a mixing chamber. Some examples of mixing chambers can include a 6L mixing chamber that is cylindrical, 5.5 inch diameter and 16.5 inches long, with an inlet at one end and an outlet at the other. With a combined scrubber outflow of approximately 1L/s, the 0.5 Hz ripple of FiO₂ (magnitude 1% or more) is attenuated by a factor of almost 10.

Thus three example approaches toward substantially constant FiO₂ output from the scrubber have been provided: (A) avoiding altering the outflow resistance during the switch to normoxia delivery, through control of hypoxia vent resistance; (B) avoiding exposing the scrubbers to reduced pressure by subject deep breathing, by providing sufficient reservoir volume (preferably at nearly constant volume around 0.1 psi); (C) attenuating ripple of the FiO₂ value with a mix chamber. Although these steps lead to nearly constant FiO₂ during a session, they do not account for possible output differences due to air humidity or scrubber wear. One example fourth step is to measure the output FiO₂ with reasonable accuracy. Therefore, to gain a meaningful measurement of administered FiO₂ during hypoxia the sensor can be occasionally calibrated with 10% calibration gas.

To achieve the rapid switching of subject FiO₂ which is desired for AIH, solenoid gas valves may be used. Such valves have 1 inch orifices and switch almost instantaneously. One way to use these valves is to have four. The scrubber output can be split within the manifold by a tee into two hoses, with valves on each side of the tee. One of the hoses connects to the subject mask, and one passes through some further plumbing to a vent. The valves are switched substantially simultaneously so one is closed when the other is open. Thus, these two valves allow scrubber flow to be rapidly switched from subject to vent, or the reverse. The venting plumbing resistance may be, for example, similar to the resistance of subject hose and mask, so that scrubber flow is not disturbed by the switch. When scrubber flow is disturbed by a vent line whose resistance is lower than the mask line, then output FiO₂ begins a slow change to a higher value, only to begin returning when venting ends. Since the concentration settling time is minutes, it is evident that such disturbance should be minimized for most use environments.

Similarly, the blower normoxia output, as discussed above, goes to a tee with a valve on each side. One side of the tee goes to a vent, and the other goes through a hose to the subject mask. Once again, these two valves may be switched substantially simultaneously to be held always in the opposite state. In other words, the blower output is directed either to the subject or to vent. This switching may be substantially simultaneous with scrubber switching, so at all times one of the two sources (scrubber, blower) is directed to the subject, while the other is directed to vent. The blower vent valve is not as important as the scrubber vent valve, and could be eliminated (see, for example FIG. 2).

One example feature of the described plumbing system is that the normoxia hose to the mask and the hypoxia hose to the mask may be two separate hoses within a manifold. These two hoses join right next to the mask. If they joined far from the mask, any change of gas would have to displace the internal volume of the hose before it reaches the mask. By using double hoses that join adjacent the mask, each is filled with the FiO₂ value it will be delivering, so initiating flow in either will deliver the required gas to the mask within a very small time.

The rebreathing concept for hypoxia (see, for example FIG. 4) is that subject breathing (exhaling as well as inhaling) of a fixed amount of air results in a reduction of O₂ and rise of CO₂. The CO₂ can be chemically scrubbed, and moisture can be removed by desiccants, to make breathable air of low FiO₂. When FiO₂ dips below the target 10%, external air can be admitted to maintain the desired level.

By using valves to isolate the rebreathing system during normoxia, the contents of the hoses are not altered. Then using multiple dedicated hoses to the mask, the subject experiences very rapid changes in FiO₂. During hypoxia, air is inhaled from the upper chamber and exhaled into the lower chamber (as controlled by one-way valves on mask inlet and outlet). The change of volume is accommodated by a single 3L reservoir bag. During normoxia, a blower feeds a dedicated inlet hose, and an outlet hose is allowed to vent.

The control, logging and safety for the intermittent hypoxia system (see, for example FIG. 5) can be handled through use of a computer, such as a laptop computer, for example. The computer operates the solenoid valves by sending commands to a microcontroller, such as an Arduino microcontroller, which controls a relay board. This board applies 110VAC to open the requisite valves. The microcontroller also reads the FiO₂ sensor signal and applies calibration coefficients to report FiO₂ to the computer.

The computer logs the valve commands and the FiO₂ readings. It also receives and logs SpO₂ (fingertip oximeter) oxygen and pulse readings, and possibly other physiological measures. The computer may monitor SpO₂ at any desired time interval and automatically (and/or with some degree of user input) make decisions to either terminate hypoxia or lengthen normoxia.

FIG. 6B illustrates a plot of automated switching where the transition of a change in FiO₂ is less than 1 second. The microcontroller controlled solenoid valves 104 achieve relatively fast gas switching that is repeatable throughout the breathing protocol. In particular, FIG. 6B illustrates the measured FiO₂ measured by the FiO₂ sensor during the command switch from normoxia to hypoxia, establishing that the value of the FiO₂ drops from its normoxic value and settles at its hypoxic value within one second, or within one sample point at a 1 Hz sampling rate. FIG. 6C illustrates overlaid MaxTec traces from several cycles, demonstrating cycle to cycle consistency in each interval. The hypoxia administration intervals are estimated at 150±0.45 seconds. Automation, therefore, affords reliable implementation of hypoxia intervals with fast switching times.

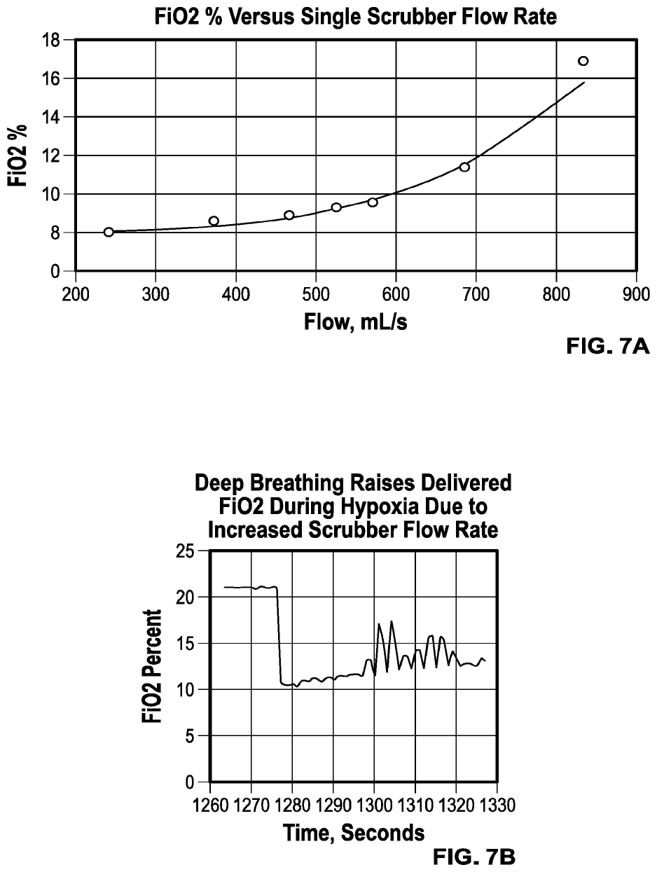

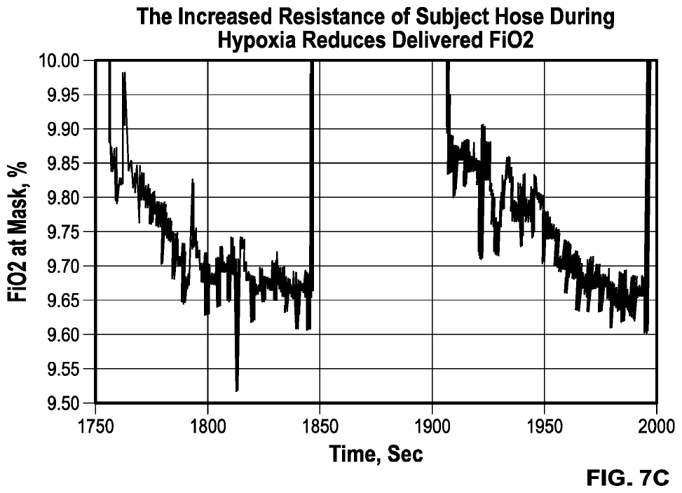

In general, oxygen concentration and flow rate are inversely related. Increasing the flow resistance increases the oxygen extraction effectiveness. For a single Hypoxico 123, for example, the oxygen concentration is plotted against flow rate in FIG. 7A. Steady flow rate is primarily adjusted by restricting the flow rate via knob on the Hypoxico 123 but varies from scrubber to scrubber. FIG. 7A illustrates a concentration vs. flow rate curve for one scrubber 112 of the system 100. Another scrubber 112 does not achieve as low a minimum FiO₂, nor is its flow rate as great when the FiO₂ is 10%. Given this limitation, the addition of a second Hypoxico 123 scrubber ensures a higher steady flow rate for hypoxic gas delivery. The additional positive pressure supplied by the cumulative flow rate of both scrubbers 112 is enough to supply sufficient gas delivery for normal respiration. Very low FiO₂ is possible only at low flow rates. At the therapeutic target of 10%, about 600 mL/s was delivered by the scrubber.

Flow rates are significantly affected by flow restrictions imposed by the respiratory behavior of the participant. Deep inhalations imposed by the participant are extreme enough to deplete the 2-liter gas reservoir bags and therefore pull extra flow through the Hypoxico 123. FIG. 7B demonstrates one such case of when the subject's breathing caused increases in delivered O₂ concentration. Upon switching to hypoxia, the first few subject inhalations did not deplete the reservoir bags but reduced the scrubber outlet pressure slightly. This resulted in a modest increase in flow of FiO₂. With further deep inhalation, the reservoir bags were fully evacuated, resulting in the subject generating suction on the scrubber, thereby increasing the FiO₂. In such an instance, the subject may report that it was qualitatively difficult to inhale.

FIG. 7C illustrates FiO₂ during gentle-breathing hypoxia of a subject, with a magnified vertical scale. As shown in FIG. 7C, the supplied FiO₂ dropped almost 0.3% in 40 seconds, implying a little more flow restriction during hypoxia. Conversely, during the normoxia period, the output flows freely to atmosphere with less flow resistance dude to no additional CPAP tubing and fittings. This reduction in flow resistance causes the FiO₂ to rise during normoxia. Adding flow restriction to the ventilated normoxia may prevent this slight drift.

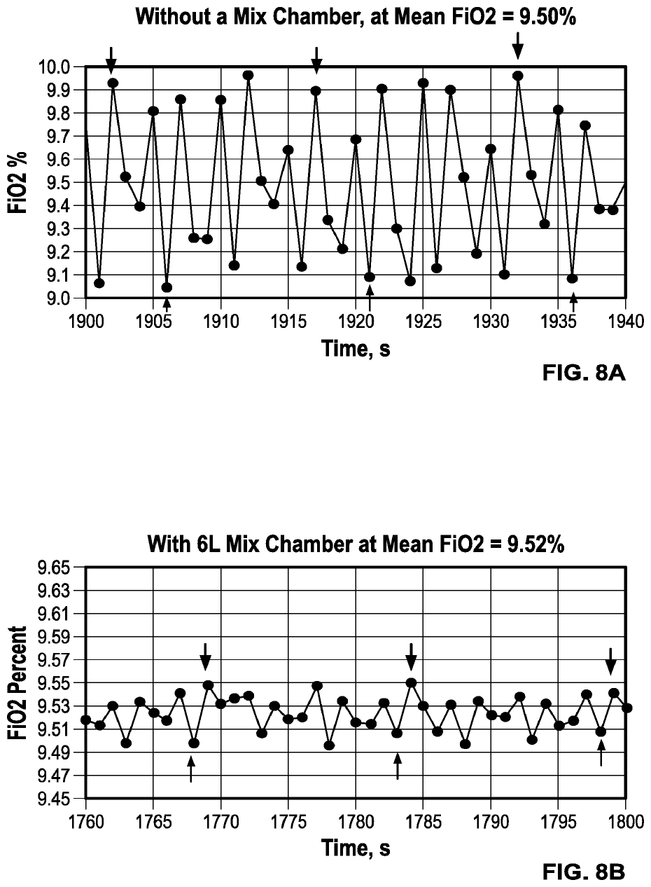

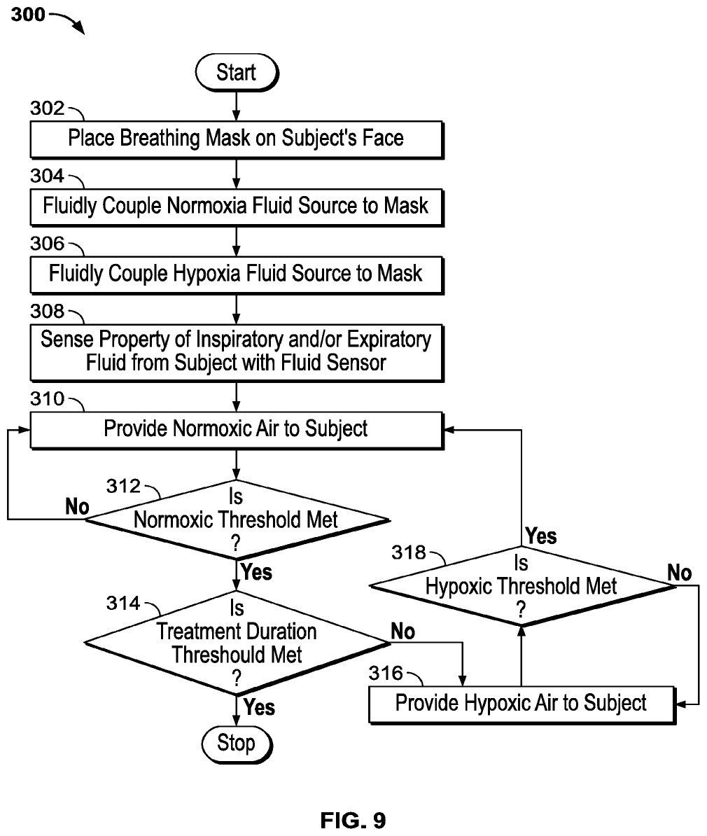

In some non-limiting examples, steady state hypoxia delivery provided by the Hypoxico 123 is not a constant 10% but fluctuates between values of 9.5% and 10.8%. This output is oscillatory as illustrated in FIG. 8A and is composed of both high frequency and low frequency components. In some examples, high frequency can correspond to 0.5 Hz and each small peak on the plot, visible as breathing of the surge bladders. In some examples, low frequency can correspond to 1/15 Hz and periodic high plotted peaks. In some cases, these recordings were revealed using the Presens sensor as the rapid ripples are unable to be detected by the slower electrochemical Maxtec sensor. Variations in the peak to peak amplitude of the oscillatory output seem to be much greater at higher O₂ settings. For example, for a target FiO₂ of 18%, the ripple amplitude is about five times greater with a clearer presentation of the described repeating pattern (not shown). In particular, FIG. 8A illustrates a ripple in FiO₂ delivered by the scrubber at a mean FiO₂ of 9.5%. The peak-to-peak ripple is about 0.9%. The arrows indicate repeating peaks and valleys.

Illustrated in FIG. 8B, the addition of a 6-liter gas mixing chamber dramatically reduces the ripple in O₂ percent delivered by the scrubber, presenting a steadier concentration to the participant. The 6-liter gas mixing chamber reduces the slope variation by approximately 90% when the target O₂ concentration is set to 9.5%. In particular, FIG. 8B illustrates the same scrubber flow as illustrated in FIG. 8A, with a 6-liter mix chamber that reduces the ripple by approximately sixteen-fold.

The systems and methods described herein generally describe an automated hypoxia delivery system that can reliably deliver a precise AIH dosing based on conventional protocols. In addition to automating specified hypoxia delivery intervals, the new system can address imperfections in the manual system including, the elimination of steady state FiO₂ concentration ripple, and ensuring a sufficient positive pressure supply without compromising O₂ concentration dosage.

The rapidly-responding 1-Hz sampling Presens FiO₂ sensor (though other sensors may be used) showed that manual switching times of FiO₂ supplied to the mask were inconsistent. For a target 90 second hypoxia interval, the best fit to concentration jump times can be a mean of 91.2 second plus or minus 1.8 seconds. For a target delivery of 60 seconds normoxia, a mean can be 57 seconds plus or minus 1.8 seconds. Lengthening of the average hypoxia intervals may be partially attributed to the lack of positive pressure in the manual system during normoxia: lacking positive pressure, subject inhalation after the normoxia interval begins will result in continued hypoxia readings at a mask sensor. Further, rapidly-settling 1-Hz sampling can show that FiO₂ of air supplied to the mask could be reliably switched in less than one second, via solenoid valves.

As discussed above, scrubber-delivered FiO₂ is inversely related to volume flow rate, which is primarily governed by a flow-restricting valve on Hypoxico 123 generators. To an occasional deep breather, one of the most noticeable defects of a single scrubber is the limited flow rate. Two scrubbers, according to aspects of the present disclosure, can provide approximately 400 and 700 mL/s when FiO₂ is at 10%. However, it should be appreciated that flow rate values may vary based on ambient temperature.

In general, sedentary adults exhibit minute ventilation of 85 to 348 mL/s (i.e., an average inspired flow rate). However, the required flow capacity may be dictated by occasional extremes, rather than averages. In some examples, the peak inspiratory flow rate expected during a typical inhalation can be up to 4 times the inspiratory average (e.g., between approximately 340 to 1390 mL/s). Examples described herein include a system having two scrubbers combined that can deliver a total flow rate (with slight positive pressure at a mask) at approximately 1100 mL/s. In some examples according to the present disclosure, additional scrubbers can be used to accommodate deeper breathers that produce a flow greater than 1390 mL/s.

In some examples where heightened breathing flow rates are expected, and two scrubbers are employed, reservoir bags may be used to avoid applying negative pressure to the scrubber output. In some examples, each reservoir bag can hold roughly 3-liters of gas when inflated, but do not deliver gas with a sustained pressure. As a result, a subject's breathing may only slightly increase in difficulty if inhaling at a faster rate than the scrubbers can deliver. Increasing the stored gas volume to multiple reservoir bags can alleviate difficulty breathing (e.g., a feeling of choking due to the increased flow resistance during deep inspiration). Even if the FiO₂ is somewhat increased by a subject's emptying, the change is likely negligible, and the subject does not feel choked of inspiratory gas. In general, when a subject breathes gently/shallowly, then FiO₂ can essentially remain constant, as desired for a reproducible hypoxia exposure.

In some implementations, devices or systems disclosed herein can be utilized using methods embodying aspects of the disclosure. Correspondingly, description herein of particular features, capabilities, or intended purposes of a device or system is generally intended to inherently include disclosure of a method of using such features for the intended purposes, a method of implementing such capabilities.

In this regard, for example FIG. 9 illustrates a method 300 for using a hypoxia delivery system. By way of example, the method 300 will be described below with reference to the hypoxia delivery system 100, although, other systems can be used. Operation 302 of the method 300 includes placing the breathing mask 106 on a subject's face 124. At operation 304, an operator may fluidly couple the normoxia fluid source 140 to the mask 106. At operation 306, the operator may fluidly couple the hypoxia source 130 to the mask 106. It should be appreciated that any of operations 302 through 206 can be completed in any order. At operation 308, a sensing sequence may be initiated and the sensor 132 may sense inspiratory and/or expiratory fluid from the subject. During the operation of the delivery system 100, normoxic air may be supplied to the subject 124 at operation 310. At a decision 312, if a threshold, such as an interval time or oxygen saturation that can be a programmable input, for example, is not met, then operation 310 continues. At decision 314, once a normoxic threshold (which can be programmable) is met, if a treatment duration threshold is met, the method 300 terminates. If a treatment duration, such as time or number of cycles is not met, then at operation 316 which provides hypoxic air to the subject 124 is initiated. At decision 318, if a threshold, such as time or blood pressure, for example, is met, then operation 310 is again initiated and the subject 124 receives normoxic air.

Each of the thresholds that may be evaluated at any of decisions 312, 314, and 318 may be user programmed into the system and can be updated based on specific needs of a patient. For example, a hypoxic threshold can be between approximately 70% and 80% FiO₂ depending on the needs of the patient. In particular, a hypoxic interval may terminate if a patient's fraction of inspired oxygen falls below 70%.

Now that particular devices, systems, and methods according to the present disclosure have been described above, a non-limiting example of an automated pressure-swing absorption system to administer low oxygen therapy for persons with a spinal cord injury will be described below.

### Methods

A. Subjects

Three able-bodied individuals (2 males and 1 female) participated in an investigation. Study participants signed informed consent and could withdraw at any time. They did not have history of neurological, pulmonary, cardiovascular, and/or severe musculoskeletal impairments. Participants completed at least one of the three experimental sessions. Data collection occurred on separate experimental sessions.
B. Equipment

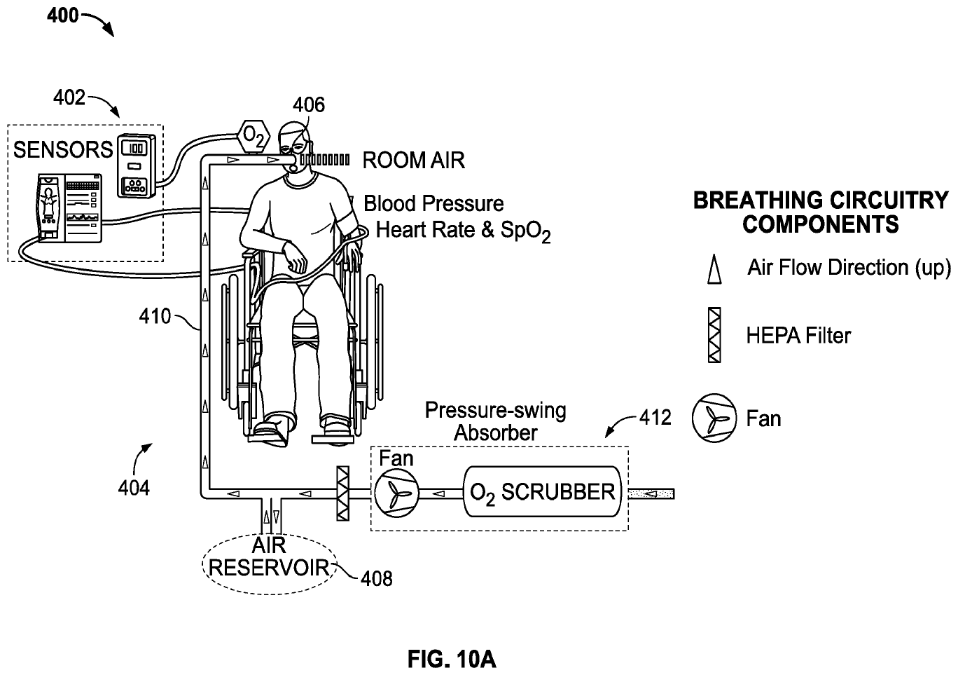

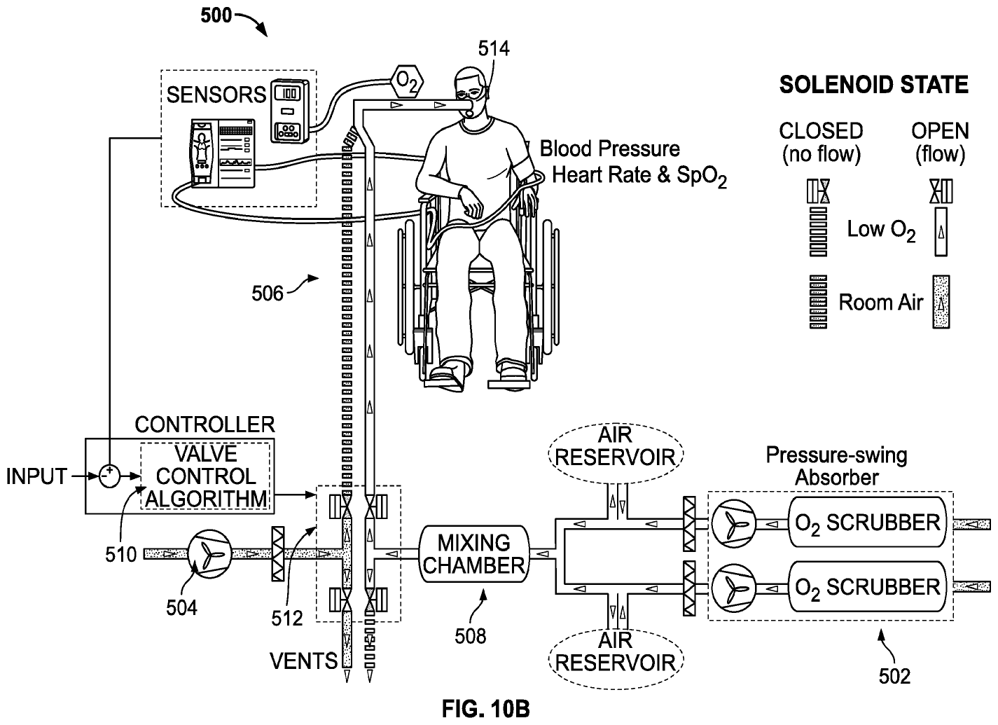

Air delivery system: As illustrated in FIG. 10, a conventional pressure-swing absorption (PSA) system 400 served as the air source for administering intermittent oxygen-depleted air treatment. The system 400 includes an onboard flow indicator and sensor 402 for manual adjustment of FiO₂ between 10.0±2.0% (low O₂) and 20.9±2.0% (room air). The system also includes a closed breathing circuit 404 to a non-rebreather mask 406 via a HEPA filter, 3-liter expandable reservoir bags 408, and approximately 3 m of clear tubing 410 per system. Expandable reservoir bags 408 provide air storage for occasional changes in inspiration rates that exceed output flow from a generator.

Air flow regulation: A microcontroller can be used to control air delivery intervals during ‘automated’ AIH protocols. During ‘manual’ AIH protocols, switching between air sources can often results in random errors in temporal sequencing of AIH. Automated valve-switching where each selected solenoid valve directs air flow using 110 VAC and 10.5 watts power consumption via a microcontroller can reduce or eliminate sequencing. The microcontroller can time a solid-state relay to distribute power to either pair of four total solenoid valves. Solenoid valves can direct air flow within the breathing circuit. Constant flows from the PSA output (hypoxia or sham) and a fan that supplies room air at positive pressure (blower) can be constantly provided. In the case of low O₂ (or sham) delivery, one valve can deliver PSA output air to the mask and the other valve can recirculate blower air to the spacious room. In the case of room air delivery, one valve can deliver blower air to the mask and the other valve redirected unused low O₂ air to the spacious indoor environment.

Room air blower: An 18.9 liter, high-flow air blower can provide continuous positive pressure air to the breathing circuit. Using a 20-ampere variable transformer set to about 20VAC blower operating speed can be regulated to produce a flow rate of 1.25 liters s⁻¹ to the mask. The room air blower provides continuous positive pressure of air to ease inspiration through the face mask.

Mask: A non-rebreather mask can be an interface between subject and air delivery system. The mask can include a one-way inlet valve that opens during inspiration of room air or delivery of positive pressure air from the generator. During expiration, the inlet valve can close, and a second one-way outlet valve can open to allow exhaled air to escape to surrounding environment. Air leakage at the mask can reduce inspired FiO₂. Inflatable padding along a face mask perimeter can reduce air leakage, and a neoprene sleeve with hook and loop strappings can secure the mask to the subject's head.

Oxygen analyzers: Two in-line O₂ analyzers can be used to monitor and record O₂ concentration within a breathing circuit. A MAX-250E galvanic, partial pressure sensor can be affixed to the breathing circuit tee connector proximal to the non-rebreather mask. The sensor can provide estimates of oxygen with a measurement range of 0 to 100%, a response time of 15 s, and full-scale linearity±1.0%. Using a MaxO₂® analyzer. Single-point calibration of this sensor can be performed at room air and ˜23° C. prior to each use.

In some instances, serial data transmitted from the analyzer (1 Hz) corresponded to changes in absolute O₂ concentration±0.2% error, with about 12-second settling time. To measure O₂ levels during rapid transitions in air sources, a high-precision fiber optic microsensor can be used. The PM-Pst7 provided estimates of O₂ concentration with a measurement range of 0 to 100%, a temporal resolution of t90=ls, a resolution of±0.01% at 1.0% O₂ and±0.05% at 20.9% O₂, and an accuracy of±0.05% at 20.9% O₂ when properly calibrated. The high-precision O₂ analyzer transmitted these digital data to a PC at 1 Hz.

Pneumotach flow sensor: A factory-calibrated linear pneumotachometer was used with an amplifier to measure air flow rate. The sensor has a linear range of±800-liters s⁻¹ and accuracy of±2.0% at room air. Flow rate can be calibrated rate using integration of the analog flow signal to a fixed 3-liter volume (calibration syringe). Flow rate signals can be acquired using a PC-based acquisition system at 1000 Hz. The sensor data estimated the magnitude and frequency of variations in air flow within the breathing circuit.

Temperature sensor: Temperatures of ambient room air and of the breathing circuit can be recorded before and during AIH and SHAM delivery protocols. A NTC thermistor was affixed adjacent to the O₂ analyzer during recordings. The sensor provided a measurement range of −50 to 99° C., a resolution of 0.1° C., and accuracy of±2.0° C. A sensor can estimate the magnitude of variations in air temperature at the face mask during AIH and SHAM treatment protocols.

Cardiopulmonary Monitor: To ensure safety, a stand-alone cardiopulmonary monitoring system recorded participant blood O₂ saturation (SpO₂), and heart rate (HR) every second, and blood pressure (BP) every five minutes. During ‘automated’ AIH delivery, a custom PC-based control software was used to acquire these parameters at 1 Hz via universal serial bus (USB).
C. Protocols

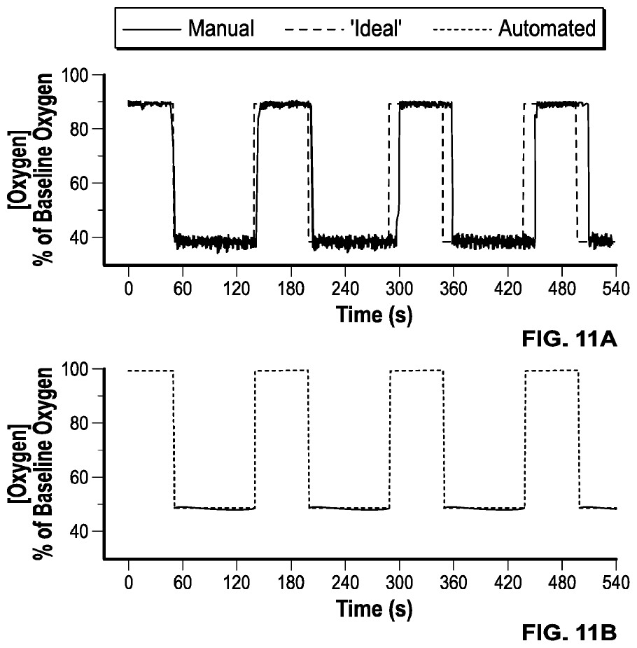

Manual delivery: The ‘manual’ AIH protocol requires a trained administrator to physically control the supply of air to a participant's non-rebreather facemask. “Manual” can be referred to as the use of a protocol administrator to ensure the air delivery system hose is either physically connected to or disconnected from the facemask at alternating time intervals (FIG. 11A). When an administrator connected the air generator to the facemask, the generator served as air source. When the administrator disconnected the air generator from the mask, the room served as air source, with flow driven by subject inspiration. An episode of AIH consisted of 60 s or 90 s bouts of generator air (nominally 10.0% or 20.9% O₂ concentration) and 60 s interval of room air (nominally 20.9% O₂ concentration). A sequence of AIH consisted of 15 episodes of both generator and room air intervals.

Automated delivery: The ‘automated’ delivery system provides alternating air delivery without the need for a trained administrator (FIG. 10B). The system consisted of a timed solenoid-valve system that directed air alternately from the air delivery system or blower to the non-rebreather facemask. A pair of PSA generators served as the low O₂ (AIH) or ambient air (SHAM) source. The air blower operated as a source of positive-pressure room air during the 60 s intervals. As shown in FIG. 11B, these sources suppled air via a common breathing circuit. Similar to the ‘manual’ delivery protocol, the circuit included HEPA filters, a set of 3-liter expandable reservoir bags, and ˜3m of clear tubing for each generator. The automated protocol also used a 6-liter mixing chamber to help reduce oscillations in FiO₂ during steady-state hypoxic air flow from the generators.

To automate air delivery, a stand-alone AIH delivery application can be used that uses the C# programming language. As opposed to ‘manual’ switching between low O₂ and room air sources, this software application provided a user interface to command switching protocols to the airflow controller. The control software required USB-interface between the microcontroller and a laptop PC running, for example, the Windows 10® operating system. The application provided protocol adjustments, as well as acquired dose timing and physiological data to ensure safety and efficacy. The application can enable users to 1) configure physiological recordings from a patient monitor, 2) adjust duration, and frequency of low O₂ intervals, 3) establish safety limits that truncate or terminate low O₂ exposure, and 4) save/load person-specific protocol settings. Collectively, these features provide safe, consistent, and flexible AIH delivery options that are not available in current ‘manual’ systems.

### Safety Protocols

Safely and consistently administering AIH treatment protocols is of highest priority. Regardless of delivery system, participant comfort and attention was monitored, as well as, their SpO₂, HR, and BP before, during, and after AIH delivery. For ‘manual’ AIH delivery protocols, a 75% SpO₂ safety limit was implemented that prompted trained personnel to remove low O₂ hosing from a participant's facemask until SpO₂ levels returned to baseline levels. This required ‘manual’ switching between air sources based on visually inspected SpO₂ readings. The ‘automated’ delivery system used real-time data acquisition and recording of SpO₂, HR, and BP to eliminate the possibility of human switching and recording errors. In cases where SpO₂ measurements fell below 75%, the ‘automated’ delivery system provided one or more 60 s intervals of room air until SpO₂ measurements returned to >90%. In cases where systolic blood pressure exceeded 150 mmHg, the delivery system provided room air.

### EXAMPLE PROTOCOLS

### Example 1

### Quantifying Dose Timing

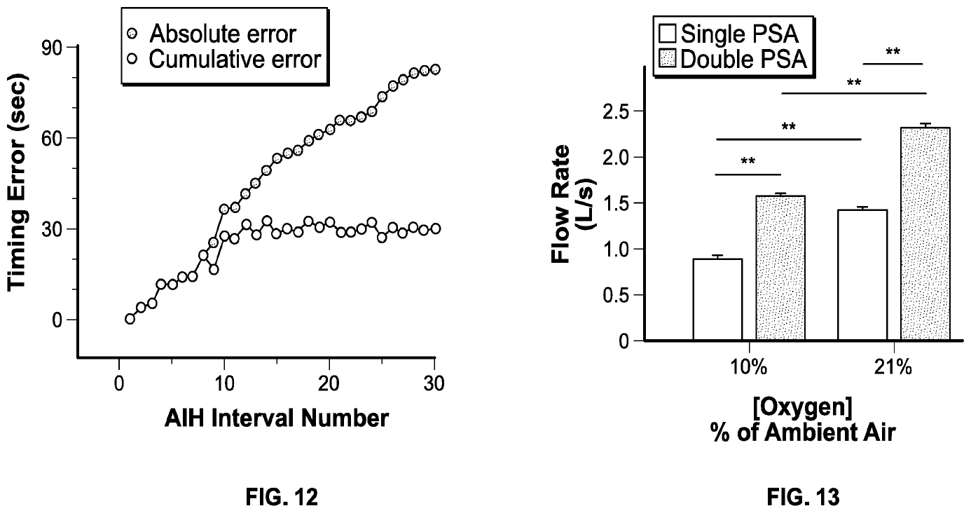

The sequence of ‘manual’ AIH treatment protocols is susceptible to human error, but the extent of this error remains unclear. In brief, the ideal dose timing of AIH consists of 15, 90 s breathing episodes of oxygen-deprived air alternating with 60 s intervals of room air. To deliver this sequence of concentration intervals to the participant, trained personnel physically attached and detached air-supply tubing to the participant's face mask. In example 1, the cumulative and absolute timing errors were measured while a trained administrator (S1) delivered a single sequence of AIH treatment (FIG. 12A).

### Example 2

### Characterizing Flow Rates

Adequate flow to the face mask is important for maintaining safe and comfortable breathing during AIH treatment protocols. The PSA systems rely on pumps (e.g., vacuum) to distribute air mixtures through the breathing circuit. However, air flow from the PSA systems are oscillatory due to alternating pump phases and other mechanisms. The pulsatile air flow propagates from the generator to the face mask and varies according to generator flow settings. In Example 2, the amplitude and frequency of fluctuations in air flow is evaluated proximal to the face mask via the ‘manual’ delivery system (one PSA with reservoir bags) that delivered 10.0% and 20.9% FiO₂. Air flow measurements were repeated using the ‘automated’ system (two PSA with reservoir bags).

### Example 3

### Characterizing Fluctuation in Oxygen Concentrations

An AIH delivery system must provide consistent levels of O₂ to the face mask to accommodate variations in breathing (i.e., deep inhales, yawns) during AIH therapy. Breathing in low O₂ often triggers episodes of deep breathing to increase tissue oxygenation, but also may exceed the available air supplied by the ‘manual’ single PSA system. Consistency in the O₂ supply examined within and between the AIH delivery systems during quiet breathing of a study participant (S2). Similar to Example 1, each system delivered a sequence of AIH. An O₂ sensor was attached proximal to the face mask and measured O₂ concentrations during the AIH sequence. S2 was then instructed to take a series of deep breaths during a single 60 s bout of low O₂. A similar protocol involved the use of an ‘automated’ AIH system.
D. Data Analyses

AIH dosing: Precise timing and amplitude of air delivery ensure safe and reliable AIH treatments. Physical switching during ‘manual’ AIH delivery is subject to dosing accuracy and timing errors. The accuracy of the delivery systems (manual, automated) was estimated to the ‘ideal’ AIH protocol defined above. Specifically, the normalized root mean square error (NRMSE) of the goodness-of-fit (GOF, eq. 1) was computed between delivery systems and the ‘ideal’ delivery protocol.

$$GOF = 100 * ( 1 - \frac{‖ y - \tilde{y} ‖}{‖ y - \overline{y} ‖} ) \qquad ( 1 )$$

where \tilde{y} is the ‘ideal’ output, y is the output from either the ‘manual’ or ‘automated’ delivery system, and y̅ is the mean of y. Timing errors corresponded to the differences between the ‘ideal’ and ‘manual’ dose time during a single AIH sequence (n=15 low O₂ episodes, n=15 room air intervals). The absolute timing error (TEabs) was computed as the absolute incremental sum of the timing errors (eq. 2).

TE_{abs}=Σᵢ₌₁³⁰|yᵢ−\tilde{y}| (2)

Flow rate: While continuous air flow to the face mask ensures user comfort and stability of AIH treatment, there is potential for fluctuations in air flow within the breathing circuit that may compromise consistency of air delivery. Thus, the extent to which frequency, amplitude, and range of flow rate change was quantified during steady-state room air and low O₂ air delivery. The peak amplitudes and ranges in measured flow rate were compared between the AIH systems (manual', ‘automated’) and FiO₂ (low O₂ at 10.0%, ambient O₂ at 20.9%).

Oxygen concentrations: Consistent and reliable FiO₂ to the face mask is necessary to maintain safe and accurate AIH treatments. Fluctuations in air flow from the PSA systems contribute to fluctuations in O₂ concentrations during AIH protocols, but the magnitude of these fluctuations remains unclear. The relationship between air flow to the face mask and oxygen-deprived air generated from the breathing circuit of the ‘manual’ and ‘automated’ delivery systems was quantified. A major design feature of the ‘automated’ system is the addition of a mixing chamber air from the HYP-123 generator. To examine the extent to which the mixing chamber reduced fluctuations in constant-resistance FiO₂, the mean absolute standard deviation (MAD) in fluctuations between the ‘automated’ delivery system (with and without the mixing chamber) and FiO₂ (low O₂ at 10.0%, ambient O₂ at 20.9%) was compared.

During episodes of low O₂ delivery, positive pressure from the PSA device ensures the AIH delivery system expels oxygen-rich air to the environment and delivers oxygen-depleted air to the breathing circuit. However, spontaneous deep breaths (e.g., yawn or sighs) and other patient specific ventilation patterns may disrupt this air separation due, in part, to pressure and/or the depletion of the available air supply by the single generator. Limited air flow from the ‘manual’ (single PSA) system during deep breathing contributes to increases in the inspiratory work of breathing and to negative pressure changes within the breathing circuit. The extent to which this vacuum may reduce generation of oxygen-depleted air needed to be characterized. Peak O₂ concentration generated from a single versus a double PSA system were compared while a participant (S2) performed five consecutive deep breaths at 10.0% FiO_{2.}

However, spontaneous deep breathes (e.g., yawn) may disrupt this air separation due, in part, to reduction in air volume within the breathing circuit. Limited air flow from the ‘manual’ (single PSA) system during deep breathing contributes to increases in the inspiratory work of breathing and to negative pressure changes within the breathing circuit. The extent to which this vacuum may reduce generation of oxygen-depleted air is not clear. Peak FiO₂ generated from a single versus a double PSA system was compared while a participant (S2) performed five consecutive deep breaths at 10.0% FiO_{2.}

Air temperature: Stable air temperature during AIH treatment is important for ensuring breathing comfort, as well as, for preserving SHAM blinding. However, the extent to which changes in O₂ during AIH protocols may result in differences in air temperature when administering room air and low O₂ is not known. Air temperature within the breathing circuit was measured during two sequences (30 episodes) of AIH and SHAM and quantified air temperature changes and compared these changes between the two air delivery methods.
E. Statistical Analyses

All statistical analyses were preformed using SPSS® 26 (IBM SPSS Inc, USA) and reported data as mean±1 standard error (SE). Statistical significance corresponded to a p-value <0.05. The Levene Test was used to determine homogeneity of variances between the independent factors. If variances differed (p >0.05), non-parametric tests were used. The TEabs to a null expectation of 0 (no error) was used using a one-sample t-test. The effects of transitioning between room air and low O₂ on TEabs was compared using a two-sample t-test. To compare the effects of AIH delivery systems on amplitude and range of the peak flow rate estimates, a linear mixed-model with fixed effects and Bonferroni corrections can be used for the multiple contrasts. A two-way analysis of variance (ANOVA) model provided comparison between the main effects of AIH delivery system (‘manual’, ‘automated’) and FiO₂ (10.0%, 20.9%) and their interactions on fluctuation in O₂ and temperature within the breathing circuit.

### Results

In this study, the performance of two AIH delivery systems was examined. The results include between-system comparisons for dose timing, air flow, FiO₂, and air temperature constancy.

AIH dose timing: The ‘manual’ delivery system is less accurate in administering an AIH protocol at prescribed timing intervals than the ‘automated’ delivery system. Goodness-of-fit between ‘ideal’ and ‘manual’ AIH delivery equated to 34.8% as compared to 98.1% between ‘ideal’ and ‘automated’ AIH delivery (FIGS. 12A and 12B). Reduced accuracy in the ‘manual’ system is due, in part, to physical switching errors. Manual attaching/detaching the hosing to the face mask resulted in a mean absolute timing error of 2.9±0.5s (t1,28 =6.4; p <0.001) that corresponded to a cumulative timing error of 30 s within a single AIH sequence (FIG. 12). Transition time resulted in a longer duration of low O₂ (93.6±0.7s) than the prescribed duration of 90 s (t1,14=3.6; p <0.003) and a shorter duration of room air exposure (58.4±0.5s) than the prescribed duration of 60 s (t1,13=4.7; p <0.001). Larger absolute timing errors occurred during transitions to room air (3.6±0.7s) than to low O₂ (2.1±0.4 s).

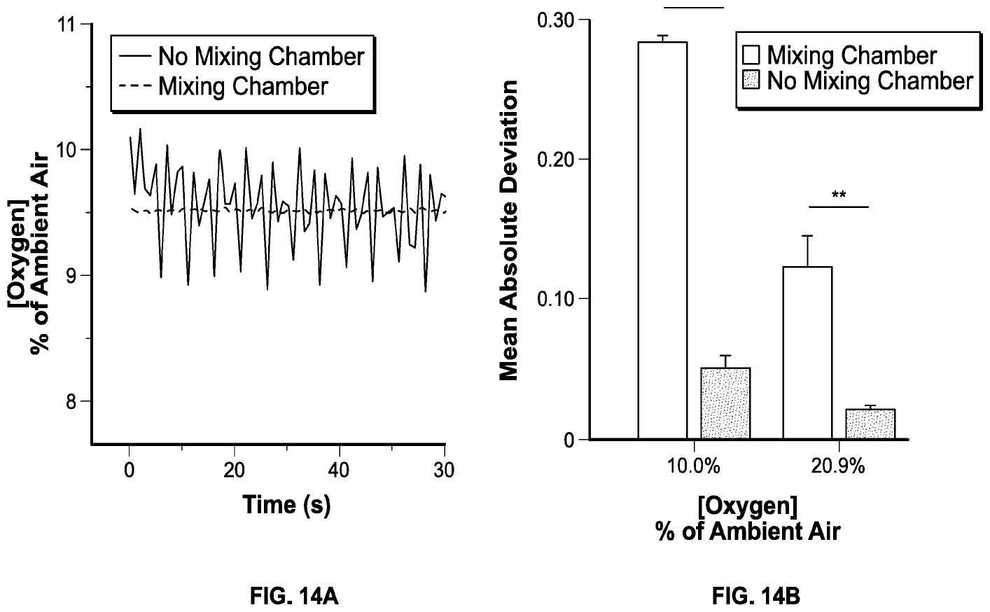

Fluctuations in flow rate: Flow rates differed between the ‘manual’ and ‘automated’ AIH delivery systems (FIG. 14). As expected, the ‘automated’ system generated greater peak and average flow rates due to the additional PSA device. At 20.9% FiO₂, peak flow rates from the ‘automated’ system reached 2.31±0.05 liters s⁻¹, which corresponded to 62.7% more flow than the ‘manual’ system (p<0.001). The higher flow rates possibly reduced one-way valve resistance at the face mask and reduced inspiratory work during breathing ‘automated’ system generated greater flow rate fluctuations at 20.9% FiO₂ (1.17±0.05 liters s⁻¹) as compared to 10.0% FiO₂ (0.90±0.03 liters s⁻¹; p <0.001), which exceeded the flow rate fluctuations within the ‘manual’ system (all p-values <0.001). The increased flow rates within the ‘automated’ system also corresponded to increased fluctuations in flow that occurred periodically at 0.4 Hz.

Variations in FiO₂: Consistency in FiO₂ is important for ensuring safe and effective AIH treatment. The reduced accuracy of AIH delivery in the ‘manual’ system is due, in part, to inherent fluctuations in FiO₂ (FIG. 14A) while the participant is not connected to the breathing circuit. During ‘manual’ air delivery, steady state FiO₂ varied±10.0% at 0.4 Hz. The inclusion of a mixing chamber within the ‘automated’ delivery system resulted in a significant improvement (±1.0% variation) in stability of air delivery at 10.0% and 20.9% FiO₂ (FIG. 14B). The mixing chamber reduced MAD to less than 5.0% during 10.0% FiO₂ and less than 1.0% during 21.0% FiO₂.

Changes in breathing volume imposed by the participants' ventilation cause breath-by-breath fluctuations in FiO₂ during AIH treatment. During quiet breathing, average flow rates are less than 0.2 liters s⁻¹. However, deep breathing may exceed the available air supplied by the single PSA system since the required flow capacity is dictated by occasional extremes, rather than averages. Deep breaths in succession, with peak flow rates exceeding 1 liter s⁻¹, depleted the ‘manual’ system's reservoir bags and reduced the PSA outlet resistance leading to less effective O₂ removal. As a result, oxygen concentrations increased 65.7% during deep breathing of low O₂ air. However, the ‘automated’ system's mixing chamber reduced the swing in FiO₂ by 28.5% during extreme deep breathing.

Temperature during AIH delivery: Significant changes in temperature within the breathing circuit were observed during AIH and SHAM protocols. The temperature fluctuated approximately±2.2° C. The ‘manual’ delivery system produced marginally higher temperatures (26.6±0.1° C.) as compared to the ‘automated’ system (26.1±0.1° C.; p <0.001). The ‘manual’ system generated a small, but significant difference (p =0.001) in temperature between AIH (26.8±0.1° C.) and SHAM (26.4±0.1° C.). However, no significant temperature differences were found between AIH (26.2±0.1° C.) and SHAM (26.0±0.1° C.) using the ‘automated’ delivery system (p =0.6). The greatest fluctuations in air temperature occurred within 45 min of system start-up. The ‘automated’ air temperatures were compared after a 45 min warm-up. The average air temperature was significantly lower (p<0.001) before warm-up at 26.6±0.1° C. versus 27.0±0.1° C. after a 45 min warm-up.

### Discussion

Mild exposure to breathing low O₂ (i.e., AIH) is a novel treatment to enhance motor function after SCI. While the ‘manual’ AIH delivery system enabled several exciting proof-of-principle experiments in humans, this technology faces design challenges that limit possible translation to clinical and home use. Here, significant inconsistencies exist in dose timing, volume flow rates, FiO₂, and to a lesser extent temperature stability of the ‘manual’ AIH delivery system.

The ‘automated’ AIH delivery system improved dose timing accuracy. The new system outperformed the ‘manual’ system by more than 63%. This is not surprising since the automated system used programmable relay switch circuits to control the state of solenoid valves on the order of milliseconds. The transition time is a major contrast to the ‘manual’ system with an administrator who switched between air sources 100x slower. Manual switching resulted in accumulation of timing errors that equated to an extra dose of low O₂ over the course of a daily exposure protocol. Fatigue may have resulted in larger errors in manual switching with time, but the results from S1 showed no positive correlation between the absolute timing error size and episode number. Timing errors are likely to vary drastically between and within administrators and treatment days. However, the ‘automated’ delivery system eliminates this variability and affords greater temporal consistency as compared to ‘manual’ AIH delivery protocols.

There are several other air delivery systems to consider, but present with deficiencies that limit their translation potential. Several commercial companies offer stand-alone PSA systems (e.g., HYP123; Hypoxico, Inc.) that transform enclosed rooms into high-altitude training experiences. While these systems are capable of achieving low O₂ levels at or near 10%, these enclosures require several minutes for transition between ambient room air and low O₂. They also do not ensure a room with uniform concentration of the prescribed air mixture. Alternatively, high-pressure gas cylinders may be a reasonable consideration since they have the capacity to deliver high precision air mixtures. Gas companies (e.g., Praxair Technology Inc., USA) provide customized gases, as well as various accessories such as check valves, fittings, regulators, alarms, and gauges. However, these cylinders require administrative operating expenses, gas handling equipment, and storage areas free from liquids, combustibles, and corrosive materials. Clinical facilities routinely accommodate these requirements, but at a premium.

Maintaining a net positive pressure of air flow to the facemask accommodates a broader range of end-users with varying breathing frequencies and tidal volumes. Average resting minute ventilation of healthy adults is between 0.09 to 0.35 liters While a single PSA meets these ventilatory demands, the required flow rate capacity needs to accommodate breath-by-breath variations in flow rates that exceed this average. A previous report showed that peak inspiratory flow rate can be nearly 4 times the average resting rate. Moreover, participants often yawn during AIH; while the physiological triggers that may induce yawns is debatable, a yawn induces rapid inspiration ˜400% greater than resting tidal volume. In either case, a second PSA with reservoir bags meets these transient increases volume and flow rate demands, as well as, ensures positive pressure at the mask for comfortable breathing.

While two PSA systems are sufficient to meet a broad range of flow rate demands, they may not be necessary. One alternative strategy to multiple PSAs is to increase the reservoir volume. Elastic reservoir bags reduce negative pressure (i.e., suction) caused when the end user's respiratory flow variations overcome the positive pressure supplied by the PSA. If the reservoir volume is emptied by several large, rapid breaths, then mask inflow is limited to the PSA flow rate. Any further inhalation is impeded, and PSA output pressure may drop by as much as 25.4 cm H₂O, for a moderate increase in flow rate, along with a rise in O₂ concentration. As was observed in one example participant (S2), deep inspiration raised the mean ventilation well above 1.0 liters s′ with peak flow rates 3 times the average, causing breathing difficulty as the inspiration rate was faster than the generators supplied. The resulting FiO₂ climbs gently while two reservoir bags are being emptied. When the bags are depleted, O₂ briefly jumps to about 17% at each deep breath. The ‘automated’ system doubles the reservoir volume to 12 liters, alleviates the breathing challenge due to higher flow resistance during deep inspiration and mitigates disturbance to O₂ concentration. Notably, even when inflated, the reservoir bags do not deliver air with a sustained pressure. If charged to approximately 7.6 cm of water pressure, bag pressure drops to 1.3 cm after delivering 0.02-0.03 liters, requiring the subject to pull the gas through the hose system with noticeable breathing difficulty.

The ‘automated’ system also reduced unwanted fluctuations in steady-state FiO₂ that were inherent to the ‘manual’ delivery system (FIG. 13). The dramatic reduction in FiO₂ fluctuations is due, in large part, to the 6-liter air mixing chamber. The chamber provided volume expansion to enable air mixing (e.g., averaging) from two generators. However, the pulsatile flow pattern of each generator is unique to the PSA mechanism and was not eliminated. Changes in flow patterns were perceivable by study participants, but the participants did not report discomfort. A concern for participants was the slight increases in breathing difficulty when the reservoir bags of the ‘manual’ system neared depletion after deep breathes. Nevertheless, changes in flow patterns that achieve the same average flow rate may be considered in future designs to further ensure that sham deliveries retain similar features as low O₂ delivery.

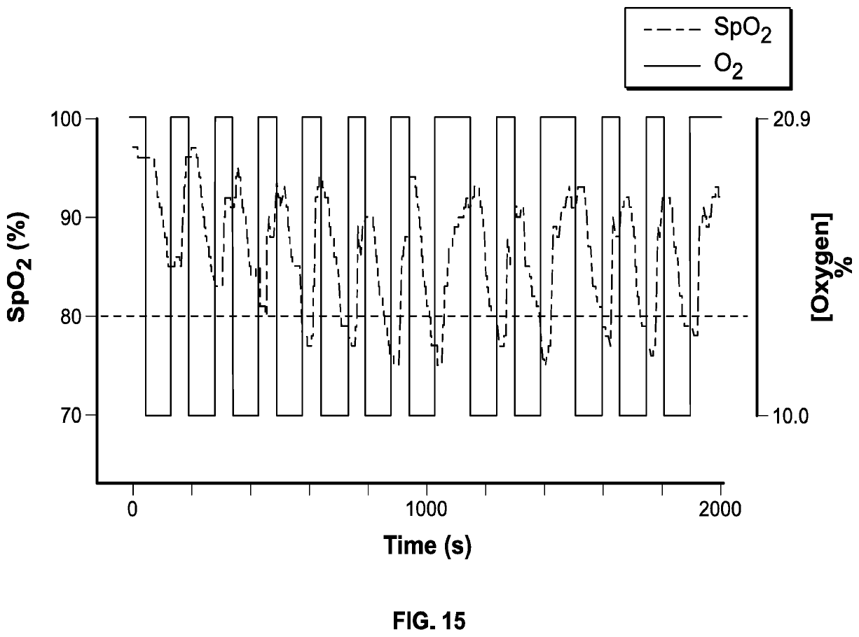

In this example, a stand-alone graphical user-interface to prescribe, monitor, and log dosing parameters and physiological data was implemented during AIH treatments. The user-interface enabled the administrator to preset the level of threshold detection for SPO₂, HR, and BP parameters for end-user safety. Thus, the AIH protocol does not require an administrator to disconnect low O₂ sources in response to cycle-to-cycle variations in the end-user's de-saturation and re-saturation rates in addition to keeping track of timing intervals. Further, override commands are implemented for the administrator to immediately stop low oxygen delivery at any point in the cycle if deemed necessary. Automated monitoring and logging of the end-user's vitals eliminates the possibility of adverse events resulting from prolonged exposures to low O₂ even when the administrator fails to override the automated switching. FIG. 15 plots representative SPO₂ data logged with the automated system from an able-bodied subject (S3) during a full 15 cycle AIH delivery. Automated extensions in room air delivery can be observed in cycle 7 when S3 did not re-saturate past the 80% SPO₂ safety threshold. Further, the user interface allows the flexibility to quickly manipulate the low oxygen and room air interval timing for studies that may require alternative dosing parameters. Together, these user-interface features simplify the experimental protocol setup while supplementing safety monitoring of the end user.

### Conclusion

An ‘automated’ AIH delivery system that confers several advantages over the previous ‘manual’ AIH system is described. The automated system 1) incorporated digital control to automate precise interval timing for temporal consistency 2) eliminated large fluctuations in delivered O₂ concentration to increase accuracy, 3) added physiological monitoring via closed loop feedback to increase safety, 4) added of continuous positive pressure to accommodate end user respiratory variations and breathing comfort, and 4) implemented a stand-alone graphical user interface for prescribing personalized treatment protocols, as well as, logging physiological responses. Together, these new features provide a consistent, safe, and flexible AIH delivery system for the development of new AIH treatment protocols.

### Additional Description of Figures

FIG. 10 illustrates a block diagram that depicts the ‘manual’ acute intermittent hypoxia (AIH) delivery system for humans (A). The system includes a single pressure-swing absorber 412 that generates and distributes low oxygen air through a human breathing circuit 404 manually connected/disconnected to the end-user's face mask 406 during AIH treatment. The dashed line denotes room air not supplied to humans during low O₂ breathing. White open triangles point along the direction of air flow. An O₂ sensor 402 near the face mask records the percentage of O₂ within the air mixture. A stand-alone patient monitor displays heart rate (HR), blood O₂ saturation (SpO₂), and blood pressure (BP) for safety.

In FIG. 10B, a block diagram depicts the ‘automated’ AIH delivery system 500. The system 500 includes double pressure-swing absorbers 502 that generate and distribute low O₂ air and a blower 504 that distributes room air through a breathing circuit 506 and a gas mixing chamber 508 that reduces fluctuations in steady state O₂ concentration. A microcontroller board 510 controls two pairs of one-way solenoid valves 512 that route air from either blower 504 or absorbers to a face mask 514. As shown, dashed lines denote air not supplied to a human. A patient monitoring unit acquires HR, SpO₂, and BP for real-time feedback to a microcontroller that maintains AIH protocols within safe limits.

FIGS. 11A and 11B illustrate quantifying temporal accuracy during an acute intermittent hypoxia (AIH) delivery protocol of 90 s breathing bouts of low O₂ with 60 s intervals of breathing ambient room air. FIG. 11A depicts time-dependent changes in relative O₂ (solid, black line) from the ‘manual’ AIH delivery system as compared to the ‘ideal’ AIH protocol. FIG. 11B depicts time-dependent changes in relative O₂ from the ‘automated’ AIH delivery system (dashed line) as compared to the ‘ideal’ AIH protocol (solid line).

FIG. 12 depicts cumulative temporal errors from S1 who administered a single sequence of AIH with the ‘manual’ delivery system. The plot with white-filled circles indicate a cumulative positive temporal error that corresponds to overall delay in the trained administrator (S1) who disconnected the tube from the face mask of participant S3. The plot with black-filled circles indicate the cumulative absolute error (secs) over time. There was a significant absolute error in switching times (p<0.001).

FIG. 13 depicts a relationship between delivered O₂ concentrations and pressure-swing absorption (PSA) flow rate. FIG. 13 further illustrates the effects of delivery system on flow rate at low O₂ (10.0±2.0%) and room air (20.9±2.0%). The bars correspond to mean±1 standard error. The black bars correspond to the flow rate for ‘automated’ system with double PSA and white bars indicate the flow rate for ‘manual’ system with single PSA. An asterisk (*) corresponds to statistical significance at p<0.01.

FIGS. 14A and 14B depict the effects of a mixing chamber on magnitude of fluctuations in steady-state O₂ concentration. In FIG. 14A, the plots show the air delivery system without the mixing chamber that resulted in ˜1% peak-to-peak O₂ fluctuations (black trace) as compared to the delivery system with a 6L mixing chamber that resulted in ˜0.06% peak-to-peak O₂ fluctuation (gray trace). In FIG. 14B, the bars represent mean±1 standard error in mean absolute deviation of O₂ concentration within the breathing circuit. The mixing chamber (black bars) significantly reduced O₂ deviations during room air and low O₂ as compared to no mixing chamber (white bars). Greater O₂ deviations occurred during low O₂ as compared to room air. Asterisks indicate significant difference (p<0.05).

FIG. 15 depicts temporal changes in blood oxygen saturation (SpO2) of a research participant (S3) during a single sequence (N=15 episodes) of acute intermittent hypoxia (AIH). The gray trace corresponds to the change in SpO₂ levels during repetitive breathing bouts at 10.0% and 20.9% O₂ (black trace). Broken black horizontal trace indicates an 80% SpO₂ safety threshold. The ‘automated’ system delivers room air when SpO₂ dips below a threshold, such as 80%, for example, during low O₂ and extends room air intervals when SpO₂ values remain below the threshold. The threshold can be user defined and adjusted based on individual need.

Within this specification aspects have been described in a way which enables a clear and concise specification to be written, but it is intended and will be appreciated that aspects of the present disclosure may be variously combined or separated without parting from the invention. For example, it will be appreciated that all preferred features described herein are applicable to all aspects of the invention described herein.

Thus, while the invention has been described in connection with particular aspects and non-limiting examples, the invention is not necessarily so limited, and that numerous other examples, uses, modifications and departures from the examples and uses are intended to be encompassed by the claims attached hereto. The entire disclosure of each patent and publication cited herein is incorporated by reference, as if each such patent or publication were individually incorporated by reference herein.

Various features and advantages of the invention are set forth in the following claims.

We claim:

1. An apparatus for providing intermittent normoxia and hypoxia intervals to a subject, the apparatus comprising:
a breathing component configured to engage the subject to deliver at least one fluid to the subject for breathing;
a normoxia fluid source coupled to the breathing component;
a hypoxia fluid source coupled to the breathing component;
a manifold fluidly coupled to the breathing component and arranged between the normoxia fluid source and the breathing component and the hypoxia fluid source and the breathing component to deliver fluid from the normoxia fluid source to the breathing component and deliver fluid from the hypoxia fluid source to the breathing component;
at least one valve configured to disrupt a flow of fluid from at least one of the normoxia fluid source and the hypoxia fluid source; and
a control system configured to:
cause the at least one valve to switch between delivery of fluid from the normoxia fluid source and the hypoxia fluid source while maintaining a positive fluid pressure at the breathing component over a full range of breathing by the subject.

2. The apparatus of claim 1, wherein the manifold includes a first hose and a second hose configured to be coupled to the normoxia fluid source and the hypoxia fluid source, respectively, and wherein fluid is injected into at least one of the first hose and the second hose at a flow rate that is greater than a breathing flow rate of the subject.

3. The apparatus of claim 1, wherein the normoxia fluid source includes a blower that intakes ambient air.

4. The apparatus of claim 1, wherein the hypoxia fluid source includes a mix chamber that is fluidly coupled with the manifold to reduce a ripple in a fraction of inspired oxygen received by the subject during hypoxia.

5. The apparatus of claim 1, wherein the hypoxia source includes a plurality of reservoir bags that is fluidly coupled to the manifold to provide a temporary increased hypoxia inspiration rate.

6. The apparatus of claim 1, wherein the hypoxia source includes at least one of a low-O₂-concentration, O₂ source or at least one oxygen scrubber fluidly coupled to the manifold to deliver the hypoxia fluid to the breathing component.

7. The apparatus of claim 1, wherein hypoxia fluid source is configured provide a range of prescribed O₂ concentrations under positive pressure.

8. The apparatus of claim 1, wherein the breathing component includes a mask that comprises a fluid sensor configured to measure a fraction of inspired oxygen at the mask.

9. The apparatus of claim 1, wherein the control system is further configured to switch the at least one valve so that the normoxia fluid source is fluidly coupled to the breathing component if the fraction of inspired oxygen is below 70%.

10. The apparatus of claim 1, further comprising at least one of a temperature sensor or a humidity sensor configured to monitor a temperature or humidity of fluid delivered to the subject and wherein the control system is configured to receive feedback from the at least one of the temperature sensor or the humidity sensor and control operation of the apparatus based on the feedback.

11. The apparatus of claim 1, wherein the control system further comprises a user interface configured to receive at least one of operational parameters, physiological parameters, or dosing intervals, and control operation of the apparatus based on the at least one of operational parameters, physiological parameters, or dosing intervals.

12. The apparatus of claim 1, further comprising at least one physiological sensor configured to monitor the subject and provide physiological feedback to the control system based on monitoring of the subject.

13. The apparatus of claim 12, wherein the control system is further configured to cause the at least one valve to actuate based on the physiological feedback and wherein the physiological feedback includes at least one of heart rate, oxygen saturation level, or blood pressure.

14. The apparatus of claim 12, wherein the control system further includes a user interface configured to receive safety thresholds and compare the physiological feedback with the safety thresholds to control operation of the apparatus to maintain the subject within the safety thresholds.

15. The apparatus of claim 1, further comprising at least one of:
a filter configured to filter fluid prior to delivery to the breathing component or filter fluid exhaled by the subject;
a disposable interface of the breathing component forming a mask to engage the subject; or
a backflow control system.

16. The apparatus of claim 1, wherein the control system is programmable to provide a range of prescribed oxygen levels to the subject.

17. The apparatus of claim 1, wherein the normoxia fluid source includes an oxygen concentration between 19% and 23% or the hypoxia fluid source includes an oxygen concentration between 8% and 12%.

18. The apparatus of claim 1, further comprising:
a normoxia fluid line connecting the normoxia fluid source to the breathing component;
a hypoxia fluid line connecting the hypoxia fluid source to the breathing component; and
wherein the normoxia fluid line and the hypoxia fluid line are separate and distinct fluid lines with no shared fluid flow paths.

19. A hypoxia delivery system comprising:
a breathing component configured to engage a face of a subject;
a subject monitoring system that includes a sensor configured to track a physiological parameter of the subject;
a normoxia source;
a hypoxia source;
a first hose line in fluid communication with the breathing component and the normoxia source;
a second hose line in fluid communication with the breathing component and the hypoxia source;
a valve system configured to control fluid flow from the normoxia source through the first hose line to the breathing component or from the hypoxia source through the second hose line to the breathing component;
a controller in communication with the subject monitoring system and configured to control operation of the valve system using feedback from the subject monitoring system.

20. The system of claim 19, wherein the sensor is configured to operate as a fraction of inspired oxygen sensor.

21. The system of claim 19, wherein the controller is configured to control the valve system to simultaneously switch between fluid flow from the normoxia source and fluid flow from the hypoxia source, such that fluid flows from only one of the normoxia source and the hypoxia source at a time.

22. The system of claim 21, wherein the controller is configured to control the valve system to perform switching between fluid flow between the normoxia source and the hypoxia source to create a settling time of the gas concentration delivered to the mask that is less than 1 second.

23. The system of claim 19, wherein the hypoxia source includes a first oxygen scrubber, a second oxygen scrubber, and a mix chamber, and wherein, the mix chamber is configured to maintain an output flow rate provided by the first and second oxygen scrubbers.

24. The system of claim 19, wherein the valve system includes a first solenoid gas valve dedicated to the first hose line and a second solenoid gas valve dedicated to the second hose line.

25. The system of claim 19, wherein the subject monitoring system is configured to measure at least one of a heart rate, a blood pressure, and an oxygen saturation level of the subject.

26. The system of claim 19, wherein the subject monitoring system is configured to sense at least one property of inspiratory and expiratory fluid from the subject and the controller is configured to provide at least one period of normoxia fluid supply to the subject and at least one period of hypoxia fluid supply to the subject.

27. A method of providing intermittent normoxia and hypoxia intervals to a subject, the method comprising:
securing a breathing component to the face of the subject, the breathing component including a hose manifold and at least one fluid sensor;
providing a normoxia fluid source;
connecting the normoxia fluid source to the hose manifold;
providing a hypoxia fluid source that is configured to provide a predetermined concentration of oxygen;
connecting the hypoxia fluid source to the hose manifold;
sensing at least one property of inspiratory fluid to the subject with the at least one fluid sensor; and
controlling normoxia and hypoxia control valves that are in fluid communication with the respective normoxia and hypoxia fluid sources to provide at least one period of normoxia fluid supply to the subject and at least one period of hypoxia fluid supply to the subject.

28. The method of claim 27, further comprising:
measuring at least one of a heart rate, a blood pressure, and an oxygen saturation level of the subject; and
instantaneously switching to a period of normoxia if a threshold level of the measured one of the heart rate, the blood pressure, and the oxygen saturation level is crossed.

29. The method of claim 28, wherein the period of normoxia if a threshold level of the measured one of the heart rate, the blood pressure, and the oxygen saturation level is crossed is a programmable time period.

30. The method of claim 27, wherein the hose manifold includes a hypoxia fluid and a normoxia fluid line; and
wherein the normoxia fluid line and the hypoxia fluid line are separate and distinct fluid lines.

31. The method of claim 27, wherein a positive pressure is maintained at the breathing component during the at least one period of normoxia fluid supply and during the at least one period of hypoxia fluid supply.
